(19) United States
(12) Patent Application Publication
(10) Pub. No.: US 2007/0037134 A1
(43) Pub. Date: Feb. 15, 2007

(54) Functional coupling of T1Rs and T2Rs by Gi proteins, and cell-based assays for the identification of T1R and T2R modulators

(75) Inventors: Guy Servant, San Diego, CA (US); Mark Ozeck, Agoura Hills, CA (US); Paul Brust, San Diego, CA (US); Hong Xu, San Diego, CA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SENOMYX, INC., LA JOLLA, CA (US)

(21) Appl. No.: 11/395,375

(22) Filed: Apr. 3, 2006

Related U.S. Application Data
(63) Continuation-in-part of application No. 10/770,127, filed on Feb. 3, 2004, now Pat. No. 7,022,488.
(60) Provisional application No. 60/457,318, filed on Mar. 26, 2003.
(60) Provisional application No. 60/444,172, filed on Feb. 3, 2003.

(51) Int. Cl. G01N 33/567; C12Q 1/00

(52) U.S. Cl. 435/4; 435/7.2

(57) ABSTRACT

The invention resides in part in the discovery that G proteins other than Gα₁₅ couples to T1R and T2R taste receptors, particularly Gᵢ proteins such as G_{αi}. Related to this discovery, the invention provides cell-based assay methods for identifying compounds that modulate the activity of specific T1R or T2R taste receptors or which modulate the effect of other T1R or T2R modulators on T1R or T2R activity. These assay methods preferably detect the effect of a putative T1R or T2R modulator compound on MAPK activation cAMP accumulation, or adenylyl cyclase activity or another signaling pathway regulated by Gᵢ proteins. The level of MAPK activation, cAMP accumulation or adenylyl cyclase is preferably determined by immunoassay methods that use ligands (monoclonal or polyclonal antibodies) that specifically bind an activated (phosphorylated) MAPK, cAMP, or adenylyl cyclase.

### PRIORITY INFORMATION

This application claims benefit of priority to U.S. Provisional Ser. No. 60/457,318 filed Mar. 26, 2003 and U.S. Ser. No. 60/444,172 filed on Feb. 3, 2003. Both of these applications are incorporated by reference in their entireties herein.

### FIELD OF THE INVENTION

The present invention relates to novel methods and materials for the identification of modulators, e.g., enhancers, agonists and antagonists of G protein-coupled receptors (GPCRs) involved in taste, i.e., T1Rs and T2Rs. These modulators may be used as flavor-affecting additives, e.g., in foods, beverages and medicines for human or animal consumption. More specifically, the present invention provides MAP Kinase, cAMP and adenylyl cyclase cell-based assays for the identification of modulators of GPCRs involved in taste modulation, i.e., T2Rs and T1Rs, preferably human T1Rs and T2Rs.

Further, the invention provides cell based assays, e.g., MAP Kinase, cAMP accumulation and adenylyl cyclase cell-based assays that rely on the discovery that G proteins other than gustducin and promiscuous and pernicious, G proteins such as Gα₁₅, i.e., Gᵢ proteins functionally-couple to T1Rs and T2Rs and use Gαi to transmit signals to downstream effectors.

### BACKGROUND OF THE INVENTION

The family of receptors that transmit signals through the activation of heterotrimeric GTP binding proteins (G proteins) constitutes the largest group of cell surface proteins involved in signal transduction. These receptors participate in a broad range of important biological functions and are implicated in a number of disease states. More than half of all drugs currently available influence GPCRs. These receptors affect the generation of small molecules that act as intracellular mediators or second messengers, and can regulate a highly interconnected network of biochemical routes controlling the activity of several members of the mitogen-activated protein kinase (MAPK) superfamily.

In fact, the activation of members of the mitogen-activated protein kinase (MAPK) family represents one of one of the major mechanisms used by eukaryotic cells to transduce extracellular signals into cellular responses (J. Blenis, Proc. Natl. Acad. Sci., USA 90:5889 (1993) (1); Blumer et al., TIBS 19:236 (1994) (2); Cano et al., TIBS 20:117 (1995) (8); Seger et al., FASEB J. 9:726 (1995) (4): R. J. Davis, TIBS 19:470 (1994) (5)). The MAPK superfamily consists of the p42 (ERK2)/p44 (ERK1) MAPKs and the stress-activated protein kinases, c-Jun N-terminal kinase (JNK) and p38 MAK (Robinson and Dickenson, Eur. J. Pharmacol. 413(2-3):151-61 (2001)(6)).

Mitogen-activated protein kinase (MAPKs) (also called extracellular signal-regulated kinases or ERKs) are rapidly activated in response to ligand binding by both growth factor receptors that function as tyrosine kinases (such as the epidermal growth factor (EGF) receptor) and receptors that are complexed with heterodimeric guanine nucleotide binding proteins (G proteins) such as the thrombin receptor. In addition, receptors like the T cell receptor (TCR) and B cell receptor (BCR) are non-covalently associated with src family tyrosine kinases which activate MAPK pathways. Specific cytokines like tumor necrosis factor (TNFalpha) can also regulate MAPK pathways. The MAPKs appear to integrate multiple intracellular signals transmitted by various second messengers. MAPKs phosphorylate and regulate the activity of enzymes and transcription factors including the EGF receptor, Rsk 90, phospholipase A₂, c-Myc, c-Jun and EIK-1/TCF. Although the rapid activation of MAPKs by tyrosine kinase receptors is dependent on Ras, G protein-mediated activation of MAPK also occurs through pathways dependent and independent of Ras.

Particularly, it is known that the activation of MAP/ERK kinase which is induced by GPCRs involves both of the G alpha and G beta gamma subunits and further involves a common signaling pathway with receptor-tyrosine-kinases. (Lopez-Ilasaca, Biochem. Pharmacol. 56(3): 269-77 (1998) (7)). For example, the G protein beta gamma subunit has been shown to activate Ras, Raf and MAP kinase in HEK293 cells. (Ito et al., FEBS Lett. 368(1): 183-7 (1995) (8)).

Additionally of relevance to the present invention, within the last several years, a number of groups including the present assignee Senomyx Inc., have reported the identification and cloning of genes from two GPCR families that are involved in taste modulation and have obtained experimental results that provide a greater understanding of taste biology. These results indicate that bitter, sweet and amino acid taste, also referred as umami taste, is triggered by activation of two types of specific receptors located at the surface of taste receptor cells (TRCs) on the tongue i.e., T2Rs and T1Rs (9-11) (Gilbertson et al., Corr. Opin. Neurobiol., 10(4):519-27 (2000); Margolskee, R F, J. Biol. Chem. 277(1):1-4 (2002); Montmayeur et al., Curr. Opin. Neurobiol., 12(4):366-71 (2002)). It is currently believed that at least 26 and 33 genes encode functional receptors (T2Rs) for bitter tasting substances in human and rodent respectively (11-13) (Montmayeur et al., Curr. Opin. Neurobiol., 12(4):366-71 (2002); Adler et al., Cell 100(6):693-702 (2000); Matsunami et al., Nature 404(6678):601-4 (2000)). By contrast there are only 3 T1Rs, T1R1, T1R2 and T1R3, which are involved in umami and sweet taste (14-16) (Li et al., Proc. Natl Acad Sci., USA 99(7):4692-6 (2002); Nelson et al., Nature (6877):199-202 (2002); Nelson et al., Cell 106(3):381-96 (2001)). Structurally, the T1R and T2R receptors possess the hallmark of G protein-coupled receptors (GPCRs), i.e., 7 transmembrane domains flanked by small extracellular and intracellular amino- and carboxyl-termini respectively.

T2Rs which have been cloned from different mammals including rats, mice and humans (12) (Adler et al., Cell 100(6): 611-8 (2000)). T2Rs comprise a novel family of human and rodent G protein-coupled receptors that are expressed in subsets of taste receptor cells of the tongue and palate epithelia. These taste receptors are organized in clusters in taste cells and are genetically linked to loci that influence bitter taste. The fact that T2Rs modulate bitter taste has been demonstrated in cell-based assays. For example, mT2R-5, hT2R-4 and mT2R-8 have been shown to be activated by bitter molecules in in vitro gustducin assays, providing experimental proof that T2Rs function as bitter taste receptors. (80) (Chandrasheker et al., Cell 100(6): 703 (2000)).

The present assignee has filed a number of patent applications relating to various T2R genes and the corresponding polypeptides and their use in assays, preferably high-throughput cell-based assays for identifying compounds that modulate the activity of T2Rs. These Senomyx applications i.e., U.S. Ser. No. 09/825,882, filed on Apr. 5, 2001, U.S. Ser. No. 191,058 filed Jul. 10, 2002 and U.S. Provisional Application Ser. No. 60/398,727, filed on Jul. 29, 2002 all incorporated by reference in their entireties herein. Additionally, the present assignee has exclusively licensed patent applications relating to T2R genes which were filed by the University of California i.e., U.S. Ser. No. 09/393,634, filed on Sep. 10, 1999 (recently allowed) and U.S. Ser. No. 09/510,332, filed Feb. 22, 2000, that describe various mouse, rat and human T2R sequences and the use thereof in assays for identifying molecules that modulate specific T2Rs and which modulate (enhance or block) bitter taste. These applications and the sequences contained therein are also incorporated by reference in their entireties herein.

Further, the present assignee and its exclusive licensor, the University of California, have filed a number of patent applications relating to human and rodent T1R taste receptors. Specifically, Senomyx has filed patent application Ser. No. 09/897,427, filed on Jul. 3, 2001, U.S. Ser. No. 10/179,373, filed on Jun. 26, 2002, and U.S. Ser. No. 09/799,629, filed on Mar. 7, 2001, all of which and the sequences contained therein are incorporated by reference in their entirety herein. Additionally, the University of California has filed a number of applications exclusively licensed by Senomyx including U.S. Ser. No. 09/361,631, filed Jul. 27, 1999, now U.S. Pat. No. 6,383,778, issued on May 7, 2002 and U.S. Ser. No. 09/361,652, filed on Jul. 27, 1999, which relates to cloned rat, mouse and human T1R1 and T1R2 genes and the use of the genes and corresponding polypeptides to identify T1R modulators. These University of California applications and the sequences contained therein are also incorporated by reference in their entirety herein.

The three T1R gene members T1R1, T1R2 and T1R3 form functional heterodimers that specifically recognize sweeteners and amino acids (14-16) (Li et al., Proc. Natl Acad Sci., USA 99(7):4692-6 (2002); Nelson et al., Nature (6877):199-202 (2002); Nelson et al., Cell 106(3):381-96 (2001)). Functional studies performed in HEK293 cells expressing the promiscuous G protein Gα_{15/16}, also disclosed therein have shown that the rodent and human T1R2/T1R3 combination recognizes natural and artificial sweeteners (14-16) (Li et al., Proc. Natl Acad Sci., USA 99(7):4692-6 (2002); Nelson et al., Nature (6877):199-202 (2002); Nelson et al., Cell 106(3):381-96 (2001)) while the rodent and human T1R1/T1R3 combination recognizes several L-amino acids and monosodium glutamate (MSG), respectively (14, 15) (Li et al., Proc. Natl Acad Sci., USA 99(7):4692-6 (2002); Nelson et al., Nature (6877):199-202 (2002)). These results, demonstrate that T1Rs are involved in sweet and umami taste.

Particularly, the co-expression of T1R1 and T1R3 in recombinant host cells results in a hetero-oligomeric taste receptor that responds to umami taste stimuli. Umami taste stimuli include by way of example monosodium glutamate and other molecules that elicit a “savory” taste sensation. By contrast, the co-expression of T1R2 and T1R3 in recombinant host cells results in a hetero-oligomeric sweet taste receptor that responds to both naturally occurring and artificial sweeteners. As with T2Rs, T1R DNAs and the corresponding polypeptides have significant application in cell and other assays, preferably high throughput assays, for identifying molecules that modulate T1R taste receptors; particularly the T1R2/T1R3 receptor (sweet receptor) and the T1R1/T1R3 receptor (umami receptor). T1R modulators can be used as flavor-affecting additives in foods, beverages and medicines.

The patents and patent application referenced above, which are incorporated by reference in their entirety herein, disclose a number of assay methods, including cell-based high throughput screening assays for identifying T1R and T2R agonists and antagonists. However, notwithstanding what is disclosed therein, novel and improved assays for identifying T1R and T2R agonists and antagonists are still needed. In particular other high throughput assays that provide for the rapid and accurate identification of T1R or T2R agonists and antagonists would be beneficial. Also, a greater understanding of what conditions and materials yield functional T1Rs and T2Rs and assays based on this greater understanding would further be beneficial.

### OBJECTS OF THE INVENTION

Toward that end, it is an object of the invention to provide a greater understanding of the means by which T1Rs and T2Rs functionally couple to G proteins and their signaling pathways.

More particularly, it is an object of the invention to identify G proteins other than Gα₁₅ and gustducin (Gᵢ proteins) which functionally couple to GPCRs involved in taste, i.e., T1Rs and T2Rs.

It is specifically an object of the invention to provide assays, preferably cell-based assays which exploit the discovery that T1Rs and T2Rs functionally couple to Gᵢ proteins, e.g. Gαᵢ.

Thus, it is an object of the invention to provide cell-based assays for identifying T1R and T2R modulators that use techniques which assay the effect of putative modulator on Gαᵢ signaling pathways.

It is a more specific object of the present invention to provide cell-based assays for identifying T1R and T2R modulators that use techniques which assay the effect of a putative T1R or T2R modulator on at least one of MAPK activity, cAMP accumulation and adenylyl cyclase activity.

More specifically, it is an object of the invention to provide novel cell-based assays for identifying T1R and T2R agonists or antagonists or enhancers that modulate MAPK activation independent of PLC activation.

It is another specific object of the invention to provide cell-based assays for identifying T1R and T2R modulators that use techniques which assay the effect of said putative modulators on Gαᵢ signaling pathways that affect downstream effectors including but not exclusive to cAMP and MAPK.

It is another specific object of the invention to provide cell-based assays for identifying T1R or T2R modulators comprising:
(i) contacting a eukaryotic cell that stably or transiently expresses at least one T1R or T2R and a G protein that functionally couples therewith, e.g., Gαi with a putative T1R or T2R modulator compound;
(ii) assaying the effect of said putative modulator compound on at least one of MAPK activation, cAMP or adenylyl cyclase activity; and
(iii) identifying whether said compound is a T1R or T2R agonist, antagonist or allosteric modulator compound based on whether it modulates the amount of activated MAPK, intracellular levels of cAMP or adenylyl cyclase activity that is expressed by said eukaryotic cell.

It is another specific object of the invention to provide novel cell-based assays for identifying compounds that modulate the effect of a known T1R or T2R activating compound, e.g., a known sweetener, umami or bitter compound comprising:
(i) contacting a eukaryotic cell that stably or transiently expresses at least one T1R or T2R and a G protein that functionally couples preferably thereto, e.g., G_{αi}, with a putative T1R or T2R modulator and with a compound that is known to activate at said least one T1R or T2R, wherein said compound and said putative agonist or antagonist compound are contacted with the eukaryotic cell separately or in combination;
(ii) assaying whether said putative modulator compounds affect at least one of MAPK activation, intracellular levels of cAMP or adenylyl cyclase activity expressed by said eukaryotic cell;
(iii) identifying whether said compound is a T1R or T2R modulator compound based on whether it results in a detectable change in activated MAPK, cAMP or adenylyl cyclase activity expressed by said eukaryotic cell.

In preferred embodiments of the invention, MAPK activation will be measured using polyclonal or monoclonal antibodies that specifically recognize activated forms of MAPK, e.g., antibodies that specifically bind p42/p44 MAPK or p38 MAPK or will be measured using proximity assays (e.g., AIphaScreen™ from Packard or High Content Screening Systems (e.g., ERK, MAPK Activation HitKit™ from Cellomics).

Also, in preferred embodiments, cAMP levels are measured by immunoassay methods, optionally after cAMP accumulation is induced by the use of a compound such as forskolin.

It is a preferred object of the invention to use the subject cell-based assays, e.g., MAPK, cAMP or adenylyl cyclase assays to identify compounds that themselves elicit sweet taste by activating the T1R2/T1R3 sweet receptor or which modulate (enhance or inhibit (block)) sweet taste elicited by another compound that activates the T1R2/T1R3 sweet receptor such as saccharin, cyclamate, saccharin, D-tryptophan, monellin, xorbitol, xylitol, L-tryptophan, and other known sweeteners.

It is another preferred object of the invention to use the subject cell-based assays, preferably MAPK, cAMP or adenylyl cyclase assays to identify compounds that themselves elicit a bitter taste or which modulate (enhance or inhibit (block)) the bitter taste elicited by another compound that activates the particular T2R, e.g., cycloheximide, denotonium, quinine, lidocaine, etc.

It is another preferred object of the invention to use the subject cell-based preferably MAPK, cAMP or adenylyl cyclase assays to identify compounds that themselves elicit umami taste by activating the T1R1/T1R3 receptor or which modulate (enhance or block) umami taste elicited by another compound that activates the T1R1/T1R3 umami receptor such as a glutamate or another savory amino acid containing compound, optionally in conjunction with inosine monophosphate.

It is another object of the invention to provide T2R or T1R agonists or antagonists identified using the subject cell-based assays that monitor the effects of a compound on G_{αi} mediated signaling pathways, e.g., cAMP, MAPK and adenylyl cyclase assays.

It is still another object of the invention to use said T2R or T1R modulatory compounds as flavor-affecting additives, e.g., in foods, beverages and medicaments for human or animal consumption.

It is yet another object of the invention to produce compositions containing T2R or T1R modulatory compounds identified using the subject cell-based MAPK and cAMP assays.

It is a specific object of the invention to provide assays for identifying modulators of T1R or T2R taste receptors wherein at least one T1R to T2R is stably or transiently expressed in a cell preferably a mammalian cell line such as HEK-293, together with a Gᵢ protein that functionally couples therewith, e.g., Gαᵢ, and the modulator is identified based on its effect on Gαᵢ mediated signaling pathways that affect the expression of downstream effectors such as cAMP, MAPK and adenylyl cyclase.

### DETAILED DESCRIPTION OF FIGURES

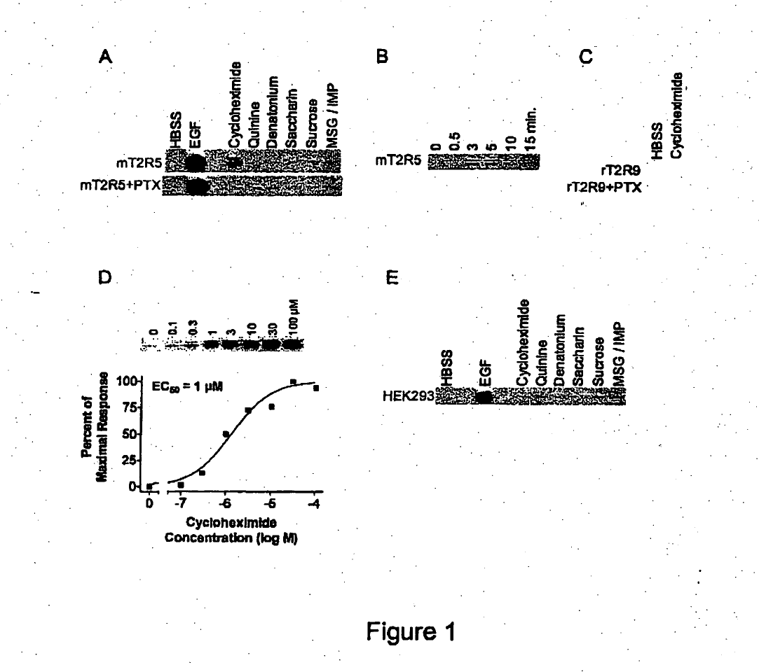

FIG. 1 contains the results of an experiment showing that mT2R5 couples to activation of ERK1/2 MAPK Panel A contains results of an experiment wherein mT2R5-expressing HEK293 cells were incubated with buffer alone (HBSS), 100 ng/mL EGF, 40 μM cycloheximide, 250 μM quinine, 2 mM denatonium, 2 mM saccharin, 100 mM sucrose, or 5 mM MSG/1 mM IMP in HBSS for 5 minutes at 37° C. Cell lysate proteins were resolved by SDS-PAGE, transferred to nitrocellulose membranes and then blotted using antibodies directed against phosphorylated ERK1/2 MAPK. PTX-treated cells were incubated with 100 ng/mL PTX overnight prior to experiment. Panel B contains an experiment that measured the course of cycloheximide-induced ERK1/2 phosphorylation in mT2R5-expressing cells. Panel C contains an experiment wherein HEK293 cells transiently pressing rT2R9 were treated as described in Panel A. Panel D contains an experiment showing the effect of increasing concentrations of cycloheximide on ERK1/2 activation. mT2R5-expressing HEK293 cells were incubated with cycloheximide diluted in HBSS (0.1 to 100 NM) for 5 minutes at 37° C. Cell lysate proteins were analyzed as described in Panel A. Bands (inset) were quantified and data were normalized to maximal stimulation of phospho-ERK1/2 MAPK (at 100 μM cycloheximide), Panel E contains an experiment wherein naive HEK293 cells were treated as described in Panel A. The results in Panels A, D and E are representative of at least 3 independent experiments. The results in Panels B and C are representative of two independent experiments.

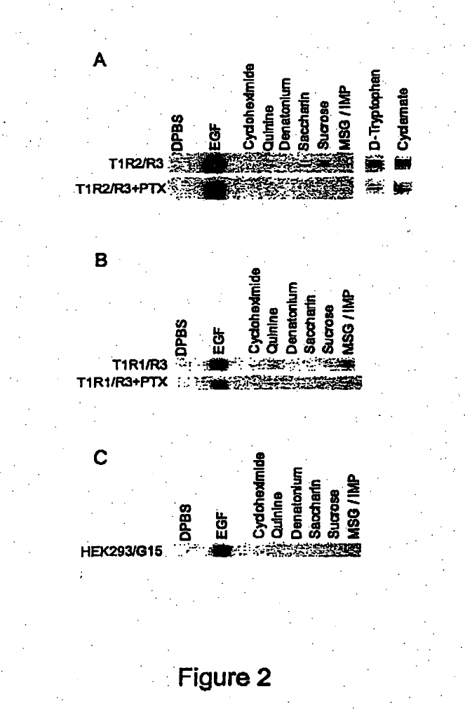

FIG. 2 contains experiments which demonstrate that hT1R2/R3 and hT1R1/R3 couple to activation of ERK1/2 MAPK. Panel A contains an experiment wherein hT1R2/R3-expressing HEK293/G15 cells incubated with buffer alone (D-PBS), 100 ng/mL EGF, 40 μM cycloheximide, 250 μM quinine, 2 mM denatonium, 2 mM saccharin, 100 mM sucrose, 5 mM MSG/7 mM IMP, 4 mM D-tryptophane and 10 mM cyclamate in D-PBS for 5 minutes at 37° C. Cell lysate proteins were resolved by SDS-PAGE, transferred to nitrocellulose membranes and then blotted using antibodies directed against phosphorylated ERK1/2 MAPK. PTX-treated cells were incubated with 100 ng/mL PIX overnight prior to the experiment. Panel B contains an experiment wherein hT1R1/hT1R3-expressing HEK293/G15 cells were treated with mifepristone to induce receptor expression (described infra) 48 hours later, cells were incubated with buffer alone (D-PBS), 100 ng/mL EGF, 40 μM cycloheximide, 250 μM quinine, 2 mM denatonium, 2 mM saccharin, 100 mM sucrose and 5 mM MSG/1 mM IMP in D-PBS for 5 minutes at 37° C. Cell lysate proteins were analyzed as described in Panel A. Panel C contains an experiment wherein naive HEK293/G15 cells were treated as described in Panel B. (Results therein are representative of at least 3 independent experiments).

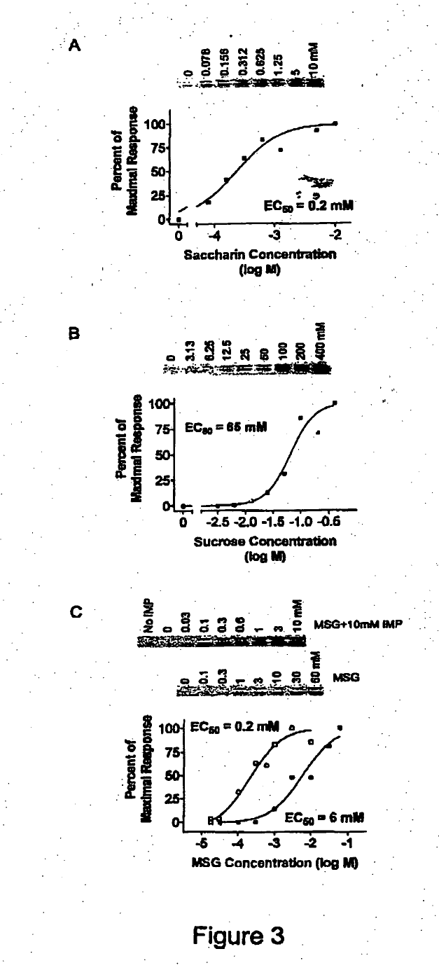

FIG. 3 contains experiments showing the effects of increasing concentrations of sweeteners and MSG on ERK1/2 activation. Panels A and B contain experiments wherein hT1R2/hT1R3-expressing HEK293/G₁₅ cells were incubated with increasing concentrations of either saccharin (Panel A) (0.078 to 10 mM) or sucrose (Panel B) (3.13 to 400 mM) for 5 minutes at 37° C. Cell lysate proteins were resolved by SDS-PAGE, transferred to nitrocellulose membranes and then blotted using antibodies directed against phosphorylated ERK1/2 MAPK. Bands (insets) were quantified and data were normalized to maximal stimulation of phospho-ERK1/2 MAPK (at 10 mM and 400 mM saccharin and sucrose respectively). Panel C contains an experiment wherein hT1R1/hT1R3-expressing HEK293/G15 cells were induced for receptor expression as described in the methods section (infra). Cells were then incubated with increasing concentrations of MSG (0.03 to 60 mM) in the absence or presence of 10 mM IMP for 5 minutes at 37° C. Cell lysate proteins were then analyzed as described in A. Bands (inset) were quantified and data were normalized to maximal stimulation of phospho-ERK1/2 MAPK (at 10 mM and 60 mM MSG). These results are representative of at least three independent experiments.

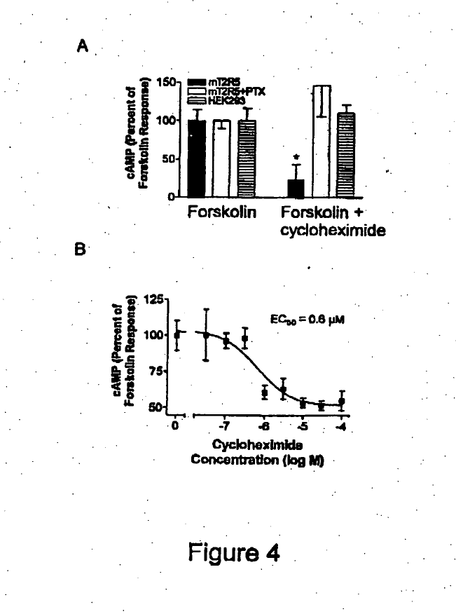

FIG. 4 contains experiments which show that cycloheximide inhibits cAMP accumulation in mT2R5-expressing cells. Panel A contains an experiment wherein mT2R5-expressing HEK293 and naive HEK293 cells were incubated with 0.7 μM forskolin and 50 μM rolipram in the absence and presence of 40 μM cycloheximide in HBSS for 15 minutes at 37° C. cAMP levels were determined as described in the methods section infra. cAMP content of mT2R5-expressing cells stimulated with buffer (0.525% DMSO in HBSS) was 5 pmol/well. cAMP content of mT2R5-expressing cells stimulated with forskolin was 73 pmol/well. Cells, were also treated with 100 ng/ml PIX for 4 hours at 37° C. and then stimulated as described above. Under these conditions the cAMP content of mT2R5-expressing cells stimulated with buffer (0.525% DMSO in HBSS) was 4 pmol/well and cAMP content of mT2R5-expressing cells stimulated with forskolin was 80 pmol/well. Panel B contains an experiment comparing the effect of increasing concentrations of cycloheximide on forskolin-induced cAMP accumulation. mT2R5-expressing HEK293 cells were incubated with 0.7 μM forskolin and 50 μM rolipram in the presence of cycloheximide diluted in HBSS (0.03 to 100 μM) for 15 minutes at 37° C. and cAMP levels were determined as described in the methods section infra. Results in Panel A correspond to the mean+/−SD of three independent experiments performed in quadruplicates. Results in Panel B are representative of three similar experiments. In the figure, * means that the result is significantly different than forskolin response, p<0.05.

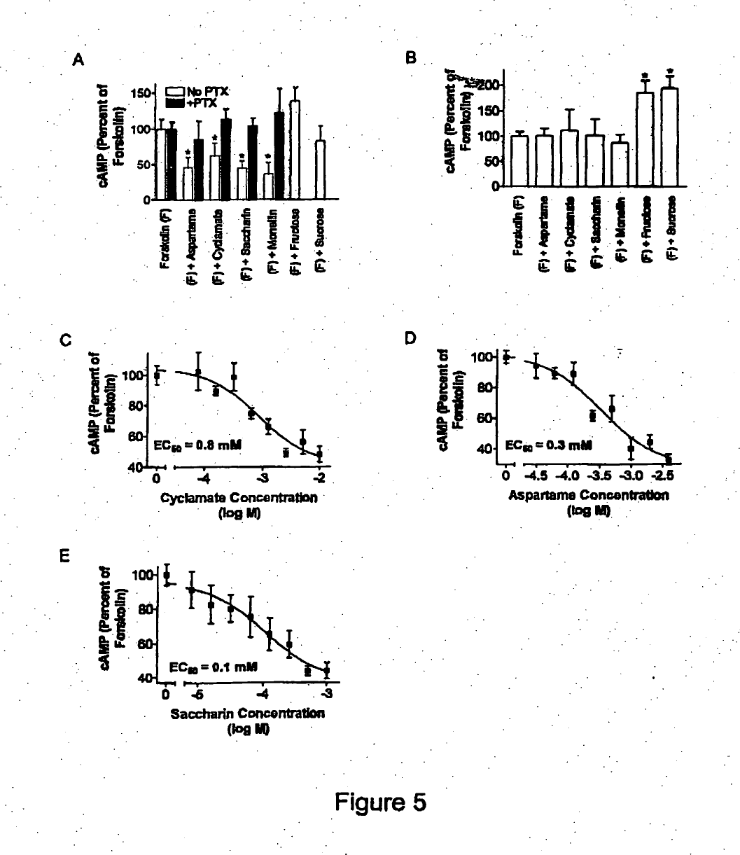

FIG. 5 contains experiments indicating that sweeteners inhibit cAMP accumulation in hT1R2/hT1R3 expressing-cells. Panel A contains an experiment wherein hT1R2/hT1R3-expressing HEK293/G15 cells were incubated with 5 μM forskolin and 50 μM rolipram in the absence and presence of either 200 mM fructose, 200 mM sucrose, 1 mM aspartame, 3 mM cyclamate, 2 mM saccharin or 50 μM monellin in D-PBS for 15 minutes at 37° C. and cAMP levels were determined as described in the methods section. cAMP content of cells stimulated with buffer (0.525% DMSO in D-PBS) was 3 pmol/well. cAMP content of mT2R5-expressing cells stimulated with forskolin was 23 pmol/well. Cells were also treated with 100 ng/ml PTX for 4 hours at 37° C. and then stimulated as described above. Under these conditions, the cAMP content of cells stimulated with buffer (0.525% DMSO in D-PBS) was 4 pmol/well and cAMP content of cells stimulated with forskolin was 149 pmol/well. Panel B shows naive HEK293/G15 cells that were treated as in Panel A. Cells stimulated with buffer (0.525% DMSO in D-PBS) was 4 pmol/well and cAMP content of cells stimulated with forskolin was 90 pmol/well. Panel C contains an experiment comparing the effects of increasing concentrations of cyclamate on forskolin-induced cAMP accumulation. Cells were incubated with of 5 μM forskolin and 50 μm rolipram in the absence or presence of increasing concentrations of cyclamate (0.08 to 10 mM). cAMP content of cells stimulated with forskolin alone was 11 pmol/well. Panel D contains an experiment comparing the effects of increasing concentration of aspartame on forskolin-induced cAMP accumulation. Cells were incubated with of 5 μM forskolin and 50 μM rolipram in the absence or presence of increasing concentrations of aspartame (0.03 to 4 mM). cAMP content of cells stimulated with forskolin alone, was 14 pmol/well. Panel E contains an experiment comparing the effects of increasing concentration of saccharin on forskolin-induced cAMP accumulation. Cells were incubated with of 5 μM forskolin and 50 μM rolipram in the absence or presence of increasing concentrations of saccharin (0.008 to 1 mM). cAMP content of cells stimulated with forskolin alone was 24 pmol/well. Results in Panels A and B correspond to the mean+/−SD of three to six independent experiments performed in quadruplicates. Results in Panels C-E are representative of three similar experiments. In the figure, * means that the result was significantly different than the forskolin response, p<0.05.

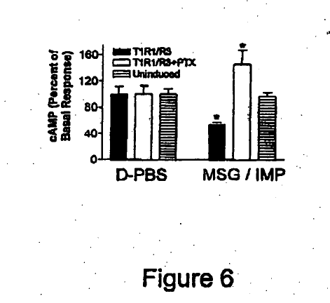

FIG. 6 contains experiments which demonstrate that MSG inhibits cAMP accumulation in hT1R1/hT1R3-expressing cells. hT1R1/hT1R3-expressing HEK293/G15 cells were induced for receptor expression as described in the methods section. (infra) Cells were incubated with 50 μM rolipram in the absence and presence of 3 mM MSG/10 mM IMP in D-PBS for 15 minutes at 37° C. and cAMP levels were determined as described in the method section. cAMP content of cells in the presence of rolipram was 120 pmol/well. Cells were also treated with 10 ng/ml PTX for 4 hours at 37° C. and then stimulated as described above. Under these conditions cAMP content of hT1R1/hT1R3-expressing cells was 95 pmol/well. Results correspond to the mean+/−SD of three independent experiments performed in quadruplicates. In the figure, * means that the result was significantly different than the forskolin response, p<0.05.

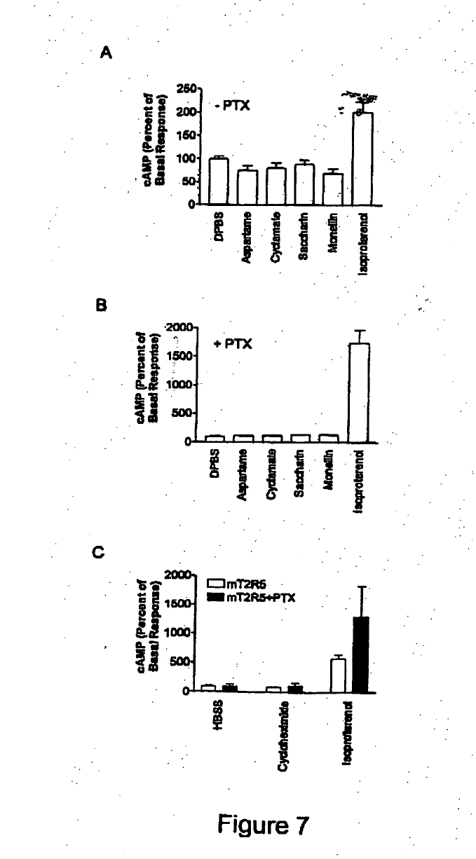

FIG. 7 contains experiments showing that mT2R5 and hT1R2/hT1R3 do not functionally couple to Gₛ. Panel A contains an experiment wherein hT1R2/hT1R3-expressing HEK293/G15 cells were incubated with 50 μM rolipram in the absence and presence of either 1 mM aspartame, 3 mM cyclamate, 2 mM saccharin, 50 μM monellin and 10 μM isoproterenol in D-PBS for 15 minutes at 37° C. and cAMP levels were determined as described in the methods section infra. Under these conditions basal level of cAMP was 2 pmol/well. Panel B contains an experiment wherein hT1R2/hT1R3-expressing cells were treated with 100 ng/ml PTX for 4 hours at 37° C. and then stimulated as described above. Under these conditions the basal level of cAMP was 1.3 pmol/well. Panel C contains an experiment wherein: mT2R5-expressing HEK293 cells were incubated with 50 μM rolipram in the absence and presence of 40 μM cycoheximide or 10 μM isoproterenol in HBSS for 15 minutes at 37° C. Under these conditions basal level of cAMP was 5 pmol/well. Cells were also treated with 100 ng/ml PTX for 4 hours at 37° C. and then stimulated as described above. Under these conditions basal level of cAMP was 4 pmol/well. Results correspond to the mean+/−SD of three independent experiments performed in quadruplicates.

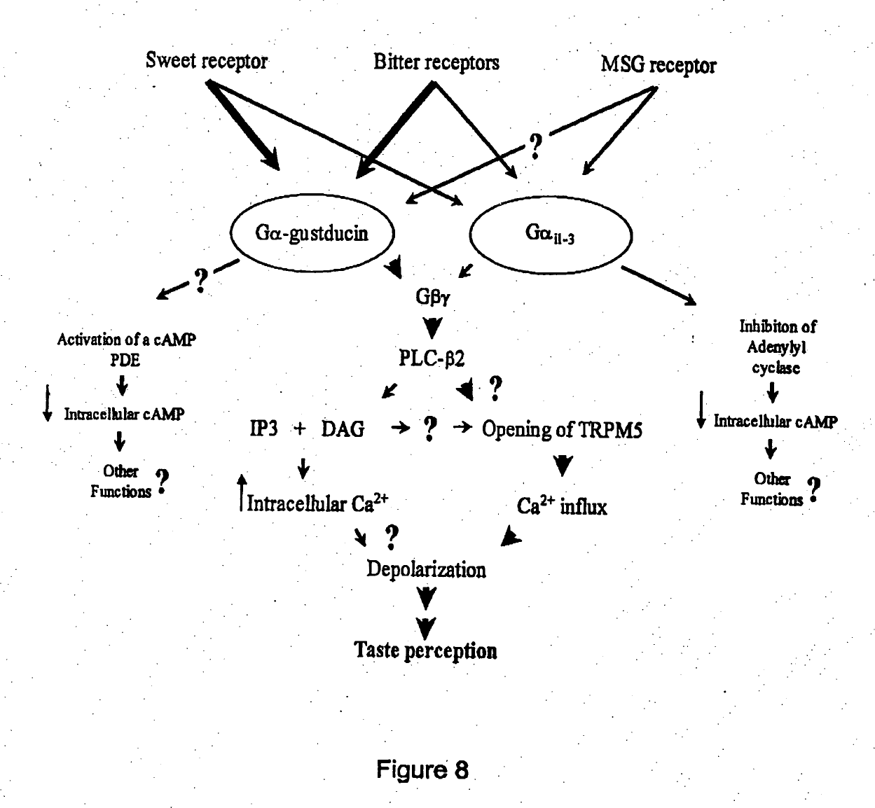

FIG. 8 contains a schematic showing how Gαᵢ is believed to complement α-gustducin signaling pathways in TRCs. Sweet and bitter receptors functionally couple to α-gustducin (thick arrows) (10, 17) Margolskee, R F, J. Biol. Chem. 277(1):1-4 (2002); Wong et al., Nature 381(6585): 796-800 (1996)). It is not known yet if the MSG (umami) receptor couples to α-gustducin but our results point to Gαᵢ as a strong candidate for its cognate G protein in TRCs. Alpha-gustducin is thought to directly couple to calcium mobilization via Gβγ and activation of PLCβ2 (9, 10) (Gilbertson et al., Curr. Opin. Neurobiol., 10(4):519-27 (2000); Margolskee, R F, J. Biol. Chem. 277(1):1-4 (2002)). Action of PLC-β2 produces two second messengers, inositol triphosphate (IP3) and diacylglycerol (DAG). IP3 triggers the release of calcium from intracellular stores. This event is not sufficient to fully depolarize TRCs (18) (Zhang et al., Cell 112(3):293-301 (2003)). DAG (19) or PLCβ2 activity itself (18) (Zhang et al., Cell 112(3):293-301 (2003)) may somehow activate a cell surface trp channel, TRPM5, leading to extracellular calcium influx in TRCs, followed by depolarization and ultimately taste perception. As disclosed in detail infra, the results of the present invention suggest that Gαᵢ is capable of complementing α-gustducin function in TRCs. Indeed, PLCβ2 is known to be activated by the Gβγ subunit of G proteins belonging to the Gᵢ family (20-24) (Li et al., Science 287(54-55):1046-9 (2000); Wu et al., Proc. Natl Acad Sci. USA 90(11):5297-5301 (1993); Katan, Biochem Biophys. Acta 1436(1-2):5-17 (1998); Smrcka et al., J. Biol Chem. 272(24):15045-48 (1993); Rhee et al., J. Biol. Chem. 272(24):15045-8 (1997)), and Gαᵢ₁₋₂ are expressed in TRCs (25, 26) (Kusakabe et al., Chem. Senses 25(5):525-31 (2000); Asano-Miyoshi Neurosci. Lett. 283(1):64 (2000)). This alternative pathway could explain the residual responsiveness of α-gustducin-deficient mouse to bitter substances and sweeteners (17, 27, 28) (Wong et al., Nature 381(6585): 796-800 (1996); He et al., Chem. Senses 27(8):719-27 (2002); Ruiz-Avila et al., Proc. Natl Acad Sci., USA 98(15):2868-73 (2001)). Current models (9, 10) (Gilbertson et al., Curr. Opin. Neurobiol., 10(4):519-27 (2000); Margolskee, R F, J. Biol. Chem. 277(1):1-4 (2002)) also suggest that α-gustducin couples to the activation of a PDE leading to a decrease of cAMP in TRCs. It is not yet known how a-gustducin may activate PDE(s). The decrease of cAMP mediated by Gαᵢ could also complement this signaling cascade. Modulation of cAMP levels in TRCs could have roles that are not yet fully defined such as defining the tone of paracrine transmission between TRCs (29) (Harness et al., J. Physiol. 543(Pt. 2):601-614 (2002)) and modulating gene expression through a balance between CREB and phosphorylated-CREB (80) (Cao et al., Neuroreport 13(10):1321-25 (2002)).

### DETAILED DESCRIPTION OF THE INVENTION

The present invention provides cell-based assays for identifying compounds that modulate, e.g., enhance, agonize or antagonize the activity of specific T1R or T2R taste receptors or that modulate the effect of another T1R or T2R activator compound preferably by assaying their effect on the expression of an activated form of MAPK, cAMP levels or adenylyl cyclase activity by a eukaryotic cell that stably or transiently expresses at least one functional T1R or T2R. In its broadest embodiment, the cell-based assays encompass the identification of T1R or T2R modulator by detecting its effect on any G_{αi} associated signaling pathway.

The invention specifically provides cell-based assays that relate to the discovery that T1Rs and T2Rs both functionally couple to G proteins other than α-gustducin or Gα₁₅, particularly Gᵢ proteins such as Gαᵢ. As discussed in detail infra, it has been shown that bitter compounds such as cycloheximide specifically activate ERK1/2 mitogen activated kinases in cells expressing a T2R and Gαᵢ and also that cycloheximide inhibits forskolin-induced cAMP accumulation. Further, it has been shown that natural and artificial sweetener compounds activate ERK1/2 in cells expressing hT1R2/hT2R13 and Gαᵢ, and that monosodium glutamate specifically activates ERK1/2 in cells expressing hT1R1/ht1R3 and Gαᵢ protein and further completely inhibits forskolin-induced cAMP accumulation in such cells; and that activation of ERK1/2 by these compounds is totally abolished by treatment with pertussin toxin. These results provide compelling evidence that the T1R and T2R receptors indeed couple and activate ERK1/2 and inhibit adenylyl cyclase through Gαᵢ.

Thus, the invention provides cell-based assays for the identification of taste modulatory compounds that rely on these discoveries. These taste modulatory compounds have potential utility as flavor enhancers or flavor additives for incorporation in foods and beverages for human or animal consumption.

### Definitions and Abbreviations

Prior to providing a detailed description of the invention, and its preferred embodiments, the following definitions and abbreviations are provided. Otherwise all terms have their ordinary meaning as they would be construed by one skilled in the relevant art.

### Abbreviations Used

Some abbreviations used in this application are set forth below.

cAMP: 3′5′-cyclic adenosine monophosphate, TRCs: Taste receptor cells, GPCRs: G protein-coupled receptors, MSG: Monosodium glutamate, PDE: phosphodiesterase; MAPK: Mitogen activated protein kinase, IMP: inosine monophosphate, PTX: pertussis toxin, EGF: Epidermal growth factor, PKC: Protein kinase C, RTKs: Receptor tyrosine kinases, PKA: Protein kinase A, ACs: Adenylyl cyclases, cNMP: cyclic nucleotide monophosphate, CREB: cAMP response element-binding protein, PLCβ2: Phospholipase Cβ2, Trp: Transient receptor potential.

“Taste cells” include neuroepithelial cells that are organized into groups to form taste buds of the tongue, e.g., foliate, fungiform, and circumvallate cells (see, e.g., Roper et al., Ann. Rev. Neurosci. 12:329-353 (1989)) (31). Taste cells are also found in the palate and other tissues, such as the esophagus and the stomach.

“T1R” refers to one or more members of a family of G protein-coupled receptors that are expressed in taste cells such as foliate, fungiform, and circumvallate cells, as well as cells of the palate, and esophagus (see, e.g., Hoon et al., Cell, 96:541-551 (1999), (32) herein incorporated by reference in its entirety). The definition of “T1R” should further be construed based on DNA and amino acid sequences disclosed in the Senomyx and University of California patent applications and publications incorporated by reference herein. (See e.g. 10-12) Members of this family are also referred to as GPCR-B3 and TR1 in WO 00/06592 as well as GPCR-B4 and TR2 in WO 00/06593. GPCR-B3 is also herein referred to as rT1R1, and GPCR-B4 is referred to as rT1R2. Taste receptor cells can also be identified on the basis of morphology (see, e.g., 31), or by the expression of proteins specifically expressed in taste cells. T1R family members may have the ability to act as receptors for sweet or umami taste transduction, or to distinguish between various other taste modalities. T1R sequences, including hT1R1, hT1R2 and hT1R3 are identified in the Senomyx and University of California patent applications incorporated by reference in their entirety herein and are provided infra, in an Appendix after the claims.

“T1R” nucleic acids encode a family of GPCRs with seven transmembrane regions that have “G protein-coupled receptor activity,” e.g., they may bind to G proteins in response to extracellular stimuli and promote production of second messengers such as IP3, cGMP, and Ca²⁺ via stimulation of enzymes such as phospholipase C and adenylate cyclase (for a description of the structure and function of GPCRs, see, e.g., Fong, T M Cells Signal. 8(3):217-224 (1996) (83) and Baldwin, et al., J. Mol. Biol. 272(1):144-164 (1997) (34). A single taste cell may contain many distinct T1R polypeptides.

The term “T1R” family therefore refers to polymorphic variants, alleles, mutants, and interspecies homologus that: (1) have at least about 35 to 50% amino acid sequence identity, optionally about 60, 75, 80, 85, 90, 95, 96, 97, 98, or 99% amino acid sequence identity to a T1R polypeptide, preferably: those identified in the patent applications incorporated by reference herein, over a window of about 25 amino-acids, optionally 50-100 amino acids; (2) specifically bind to antibodies raised against an immunogen comprising an amino acid sequence preferably selected from the group consisting of the T1R polypeptide sequence disclosed in the patent applications incorporated by reference herein, and conservatively modified variants thereof; (3) are encoded by a nucleic acid molecule which specifically hybridize (with a size of at least about 100, optionally at least about 500-1000 nucleotides) under stringent hybridization conditions to a sequence selected from the group consisting of the T1R nucleic acid sequences contained in the applications incorporated by reference in their entirety herein, and conservatively modified variants thereof; or (4) comprise a sequence at least about 35 to 50% identical to an amino acid sequence selected from the group consisting of the T1R amino acid sequence identified in the patent applications incorporated by reference in their entirety herein.

The term “T2R” refers to one or more members of a family of G protein coupled receptors that are expressed in taste cells, specifically, the tongue and palate epithelia. In particular, T2R includes the particular genes identified in the Senomyx and University of California applications relating to T2Rs incorporated by reference in their entirety herein. T2Rs are genetically linked to loci associated with bitter taste perception in mice and humans. More specifically, the term “T2R” and terms including T2R, e.g., T2R04 or T2R05 refers generally to isolated T2R nucleic acids, isolated polypeptides encoded by T2R nucleic acids, and activities thereof. T2R nucleic acids and polypeptides can be derived from any organism. The terms “T2R” and terms including “T2R” also refer to polypeptides comprising receptors that are activated by bitter compounds, and to nucleic acids encoding the same. Thus both T1Rs and T2Rs comprise different families of chemosensory GPCRs. Sequences of various T2Rs are also contained in the Appendix that precedes the claims.

G proteins are heterotrimeric proteins composed of a single α subunit complexed with the βγ dimer. Molecular cloning has resulted in the identification of 18 distinct .α. subunits, 5β subunits, and 12γ subunits. G proteins are usually divided into four subfamilies Gᵢ, Gₛ, G_{q}, and G₁₂ based on the sequence similarity of the Gα subunit. Several lines of evidence suggest that the interaction between a given GPCR and its cognate G protein involves multiple sites of contact on both proteins. All three intracellular loops as well as the carboxyl terminal tail of the receptor have been implicated. The GPCR is thought to interact with all three subunits of the G protein. As the receptor-G protein interaction can be disrupted by a number of treatments that block the carboxyl terminus, including pertussis toxin-catalyzed ADP-ribosylation of G_{α} and binding of monoclonal antibodies, the carboxy terminal region of the Gα subunit has been the most intensely investigated contact site. These studies have shown that the G_{α}, carboxy-terminal region is important not only to the interaction, but also plays a critical role in defining receptor specificity (Hamm et al., Science 241: 832-5 (1988); Osawa et al., J. Biol. Chem. 270: 31052-8 (1995); Garcia et al., EMBO 14: 4460-9 (1995); Sullivan et al., Nature 330: 758-760 (1987); Rasenick et al., J. Biol. Chem. 269: 21519-21525 (1994); West et al., J. Biol. Chem. 260: 14428-30 (1985); Conklin et al., 1993, Nature 363: 274-276; Conklin et al., Mol. Pharmacol. 50: 885-890 (1996)) (3542). Furthermore, it has been shown that peptides corresponding to the carboxy terminal region of a G_{αi} subunit can block GPCR signaling events (Hamm et al., Science 241: 832-5 (1988); Gilchrist et al., J. Biol. Chem 273: 14912-19 (1998)) (35, 43). However, prior to the present invention, it was unknown that Gᵢ proteins were capable of functionally coupling to T1Rs and T2Rs.

Topologically, certain chemosensory GPCRs have an “N-terminal domain;” “extracellular domains;” “transmembrane domains” comprising seven transmembrane regions, and corresponding cytoplasmic, and extracellular loops; “cytoplasmic domains,” and a “C-terminal domain” (see, e.g., Hoon et al., Cell, 96:541-551 (1999) (115); Buck & Axel, Cell, 65:175-187 (1991)) (44). These domains can be structurally identified using methods known to those of skill in the art, such as sequence analysis programs that identify hydrophobic and hydrophilic domains (see, e.g., Stryer, Biochemistry, (3rd ed. 1988) (45); see also any of a number of Internet based sequence analysis programs. Such domains are useful for making chimeric proteins and for in vitro assays of the invention, e.g., ligand binding assays.

“Extracellular domains” therefore refers to the domains of T1R and T2R polypeptides that protrude from the cellular membrane and are exposed to the extracellular face of the cell. Such domains generally include the “N terminal domain” that is exposed to the extracellular face of the cell, and optionally can include portions of the extracellular loops of the transmembrane domain that are exposed to the extracellular face of the cell, i.e., the loops between transmembrane regions 2 and 3, between transmembrane regions 4 and 5, and between transmembrane regions 6 and 7.

The “N-terminal domain” region starts at the N-terminus and extends to a region close to the start of the first transmembrane domain. More particularly, in one embodiment of the invention, this domain starts at the N-terminus and ends approximately at the conserved glutamic acid at amino acid position 563 plus or minus approximately 20 amino acids. These extracellular domains are useful for in vitro ligand-binding assays, both soluble and solid phase. In addition, transmembrane regions, described below, can also bind ligand either in combination with the extracellular domain, and are therefore also useful for in vitro ligand-binding assays.

“Transmembrane domain,” which comprises the seven “transmembrane regions,” refers to the domain of T1R or T2R polypeptides that lies within the plasma membrane, and may also include the corresponding cytoplasmic (intracellular) and extracellular loops. In one embodiment, this region corresponds to the domain of T1R or T2R family members. In the case of T1R family member this starts approximately at the conserved glutamic acid residue at amino acid position 563 plus or minus 20 amino acids and ends approximately at the conserved tyrosine amino acid residue at position 812 plus or minus approximately 10 amino acids. The seven transmembrane regions and extracellular and cytoplasmic loops can be identified using standard methods, as described in Kyte & Doolittle, J. Mol. Biol., 157:105-32 (1982)) (46), or in Stryer, supra (45).

“Cytoplasmic domains” refers to the domains of T1R or T2R polypeptides that face the inside of the cell, e.g., the “C-terminal domain” and the intracellular loops of the transmembrane domain, e.g., the intracellular loop between transmembrane regions 1 and 2, the intracellular loop between transmembrane regions 3 and 4, and the intracellular loop between transmembrane regions 5 and 6. “C-terminal domain” refers to the region that spans the end of the last transmembrane domain and the 0-terminus of the protein, and which is normally located within the cytoplasm. In one embodiment, this region starts at the conserved tyrosine amino acid residue at position 812 plus or minus approximately 10 amino acids and continues to the C-terminus of the polypeptide.

The term “ligand-binding region” or “ligand-binding domain” refers to sequences derived from a taste receptor, particularly a taste receptor that substantially incorporates at least the extracellular domain of the receptor. In one embodiment, the extracellular domain of the ligand-binding region may include the N-terminal domain and, optionally, portions of the transmembrane domain, such as the extracellular loops of the transmembrane domain. The ligand-binding region may be capable of binding a ligand, and more particularly, a compound that enhances, mimics, blocks, and/or modulates taste, e.g., sweet, bitter, or umami taste. In the case of T2Rs, the compound bound by the ligand binding region will modulate bitter taste. In the case of T1Rs, the compound bound by the ligand-binding region will modulate sweet or umami taste.

The phrase “heteromultimer” or “heteromultimeric complex” in the context of the T1R receptors or polypeptides used in the assays of the present invention refers to a functional association of at least one T1R receptor and another receptor, typically another T1R receptor polypeptide (or, alternatively another non-T1R receptor polypeptide). For clarity, the functional co-dependence of the T1Rs is described in this application as reflecting their possible function as heterodimeric taste receptor complexes. However, as discussed in Senomyx patent applications and publications which are incorporated by reference herein, (10-12) functional co-dependence may alternatively reflect an indirect interaction. For example, T1R3 may function solely to facilitate surface expression of T1R1 and T1R2, which may act independently as taste receptors. Alternatively, a functional taste receptor may be comprised solely of T1R3, which is differentially processed under the control of T1R1 or T1R2, analogous to RAMP-dependent processing of the calcium-related receptor. By contrast, in the case of T2Rs the eukaryotic cells used in the subject MAPK assays will preferably express a single T2R.

The phrase “modulator” or “modulatory compound” means any compound that itself affects the activity of a T1R or T2R or modulates (affects) the effect of another compound on T1R or T2R activity. Typically, modulation is determined by cell-based assays that detect the effect of a putative modulator or Gi signaling pathways, e.g., assays that detect the effect of a compound on MAPK activity, cAMP levels or adenylyl cyclase activity.

The phrase “functional effects” in the context of assays for testing compounds that modulate at least one T1R or T2R family member mediated taste transduction includes the determination of any parameter that is indirectly or directly under the influence of the receptor, e.g., functional, physical and chemical effects. It includes ligand binding, changes in ion flux, membrane potential, current flow, transcription, G protein binding, GPCR phosphorylation or dephosphorylation, conformation change-based assays, signal transduction, receptor-ligand interactions, second messenger concentrations (e.g., cAMP, cGMP, IP3, or intracellular Ca²⁺), in vitro, in vivo, and ex vivo and also includes other physiologic effects such increases or decreases of neurotransmitter or hormone release. In the present invention, the assays will generally measure the effect of a compound on MAPK activation, cAMP accumulation or adenylyl cyclase activity in cell-based expression systems whereby the T1R or T2R is functionally coupled to a Gᵢ protein such as G_{αi} and the assays are used to screen for putative sweeteners or sweet taste modulators or enhancers, umami taste modulators or enhancers, or bitter compounds or bitter taste modulators or enhancers, e.g., bitter taste blockers. Such modulators have application for incorporation in foods, beverages, pharmaceuticals, and the like for human or animal consumption.

By “determining the functional effect” in the context of assays is meant assays for a compound that increases or decreases a parameter that is indirectly or directly under the influence of at least one T1R or T2R family member, e.g., functional, physical and chemical effects. Such functional effects can be measured by any means known to those skilled in the art, e.g., changes in spectroscopic characteristics (e.g., fluorescence, absorbency, refractive index), hydrodynamic (e.g., shape), chromatographic, or solubility properties, patch clamping, voltage-sensitive dyes, whole cell currents, radioisotope efflux, inducible markers, oocyte T1R or T2R gene expression; tissue culture cell T1R or T2R expression; transcriptional activation of T1R or T2R genes; ligand-binding assays; voltage, membrane potential and conductance changes; ion flux assays; changes in intracellular second messengers such as cAMP, cGMP, and inositol, triphosphate (IP3); changes in intracellular calcium levels; neurotransmitter release, conformational assays and the like. In the present invention, the effect of a putative modulator compound will be preferably assayed based on its effect on MAPK activation, cAMP accumulation, or adenylyl cyclase activity.

“Inhibitors,” “activators,” “enhancer,” and “modulators” of T1R or T2R genes or proteins are used to refer to inhibitory, activating, or modulating molecules identified using in vitro and in vivo assays for taste transduction, e.g., ligands, agonists, antagonists, inversed agonists, and their homologues and mimetics. These compounds themselves modulate T1R or T2R activity or modulate the effect of another compound on T1R or T2R activity. In the present invention these molecules will preferably be identified using the subject cell-based MAPK or cAMP assays. In preferred embodiments, the “inhibitors” will block taste of a known bitter compound or enhance the taste of a known sweet or umami compound or compounds.

Inhibitors are compounds that, e.g., bind to, partially or totally block stimulation, decrease, prevent, delay activation, inactivate, desensitize, or down regulate taste transduction, e.g., antagonists. Activators are compounds that, e.g., bind to, stimulate, increase, open, activate, facilitate, enhance activation, sensitize, or up regulate taste transduction, e.g., agonists. Modulators include compounds that, e.g., alter the interaction of a receptor with: extracellular proteins that bind activators or inhibitor (e.g., ebnerin and other members of the hydrophobic carrier family); G proteins; kinases (e.g., homologues of rhodopsin kinase and beta adrenergic receptor kinases that are involved in deactivation and desensitization of a receptor); and arrestins, which also deactivate and desensitize receptors. Modulators can include genetically modified versions of T1R or T2R family members, e.g., with altered activity, as well as naturally occurring and synthetic ligands, antagonists, agonists, small chemical molecules and the like. Such assays for inhibitors and activators include, e.g., expressing T1R or T2R family members in cells or cell membranes, applying putative modulator compounds, in the presence or absence of tastants, e.g., sweet, umami or bitter tastants, and then determining the functional effects on taste transduction, as described above. Samples or assays comprising T1R or T2R family members that are treated with a potential activator, inhibitor, or modulator are compared to control samples without the inhibitor, activator, or modulator to examine the extent of modulation. Positive control samples (e.g. a sweet, umami, or bitter tastant without added modulators) are assigned a relative T1R or T2R activity value of 100%.

Negative control samples (e.g., buffer without an added taste stimulus) are assigned a relative T1R or T2R activity value of 0%. Inhibition of a T1R or T2R is achieved when a mixture of the positive control sample and a modulator result in the T1R or T2R activity value relative to the positive control is about 80%, optionally 50% or 25-0%. Activation of a T1R or T2R by a modulator alone is achieved when the T1R activity value relative to the positive control sample is 10%, 25%, 50%, 75%, optionally 100%, optionally 150%, optionally 200-500%, or 1000-3000% higher.

The terms “purified,” “substantially purified,” and “isolated” as used herein refer to the state of being free of other, dissimilar compounds with which the compound of the invention is normally associated in its natural state, so that the “purified,” “substantially purified,” and “isolated” subject comprises at least 0.5%, 1%, 5%, 10%, or 20%, and most preferably at least 50% or 75% of the mass, by weight, of a given sample. In one preferred embodiment, these terms refer to the compound of the invention comprising at least 95% of the mass, by weight, of a given sample. As used herein, the terms “purified,” “substantially purified,” and “isolated,” when referring to a nucleic acid or protein, also refers to a state of purification or concentration different than that which occurs naturally in the mammalian, especially human body. Any degree of purification or concentration greater than that which occurs naturally in the mammalian, especially human, body, including (1) the purification from other associated structures or compounds or (2) the association with structures or compounds to which it is not normally associated in the mammalian, especially human, body, are within the meaning of “isolated.” The nucleic acid or protein or classes of nucleic acids or proteins, described herein, may be isolated, or otherwise associated with structures or compounds to which they are not normally associated in nature, according to a variety of methods and processes known to those of skill in the art.

The term “nucleic acid” or “nucleic acid sequence” refers to a deoxy-ribonucleotide or ribonucleotide oligonucleotide in either single- or double-stranded form. The term encompasses nucleic acids, i.e., oligonucleotides, containing known analogs of natural nucleotides. The term also encompasses, nucleic-acid-like structures with synthetic backbones (see e.g., Oligonucleotides and Analogues, a Practical Approach, ed. F. Eckstein, Oxford Univ. Press (1991); Antisense Strategies, Annals of the N.Y. Academy of Sciences, Vol. 600, Eds. Baserga et al. (NYAS 1992); Milligan J. Med. Chem. 36:1923-1937 (1993); Antisense Research and Applications (1993, CRC Press), Mata, Toxicol. Appl. Pharmacol. 144:189-197 (1997); Strauss-Soukup, Biochemistry 36:8692-8698 (1997); Samstag, Antisense Nucleic Acid Drug Dev, 6:153-156 (1996)) (47-53).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating, e.g., sequences in which the third position of one or more selected codons is substituted with mixed-base and/or deoxyinosine residues (Batzer et al, Nucleic Acid Res., 19:5081 (1991); Ohtsuka et al., J. Biol. Chem., 260:2605-2608 (1985); Rossolini et al., Mol. Cell. Probes, 8:91-98 (1994)) (54-56). The term nucleic acid is used interchangeably with gene, cDNA, mRNA, oligonucleotide, and polynucleotide.

The terms “polypeptide,” “peptide” and “protein” are used: interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “plasma membrane translocation domain” or simply “translocation domain” means a polypeptide domain that, when incorporated into a polypeptide coding sequence, can with greater efficiency “chaperone” or “translocate” the hybrid (“fusion”) protein to the cell plasma membrane than without the domain. For instance, a “translocation domain” may be derived from the amino terminus of the bovine rhodopsin receptor polypeptide, a 7-transmembrane receptor. However, rhodopsin from any mammal may be used, as can other translocation facilitating sequences. Thus, the translocation domain is particularly efficient in translocating 7-transmembrane fusion proteins to the plasma membrane, and a protein (e.g., a taste receptor polypeptide) comprising an amino terminal translocating domain will be transported to the plasma membrane more efficiently than without the domain. However, if the N-terminal domain of the polypeptide is active in binding, as with the T1R or T2R receptors of the present invention, the use of other translocation domains may be preferred. For instance, a PDZ domain-interacting peptide, as described herein, may be used.

The “translocation domain,” “ligand-binding domain”; and chimeric receptors compositions described herein also include “analogs,” or “conservative variants” and “mimetics” (“peptidomimetics”) with structures and activity that substantially correspond to the exemplary sequences. Thus, the terms “conservative variant” or “analog” or “mimetic” refer to a polypeptide which has a modified amino acid sequence, such that the change(s) do not substantially alter the polypeptide's (the conservative variant's) structure and/or activity, as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity.

More particularly, “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein.

For instance, the codons GCA, GCC, GCG and GCU all encode the amino acid alanine. Thus, at every position where an alanine is specified by a codon, the codon can be altered to any of the corresponding codons described without altering the encoded polypeptide.

Such nucleic acid variations are “silent variations,” which are one species of conservatively modified variations. Every nucleic acid sequence herein, which encodes a polypeptide, also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid, which encodes a polypeptide, is implicit in each described sequence.

Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gin; ile/leu or val; leu/ile or val; lys/arg or gln or glu; met/leu or tyr or lie; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (I); 5) Isoleucine (1), Leucine (L), Methionine (M), Valine (V); and 6) Phenylalanine (F), Tyrosine (Y), Tryptophan (W); (see also, e.g., Creighton, Proteins, W.H. Freeman and Company (1984); Schultz and Schimer, Principles of Protein Structure, Springer-Verlag (1979)) (57-58). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

The terms “mimetic” and “peptidomimetic” refer to a synthetic chemical compound that has substantially the same structural and/or functional characteristics of the polypeptides, e.g., translocation domains, ligand-binding domains, or chimeric receptors of the invention. The mimetic can be either entirely composed of synthetic, non-natural analogs of amino acids, or may be a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity.

As with polypeptide's of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g. a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′dicyclohexylcarbodiimide (DCC) or N,N′-diisopropylcarbodiimide (DIC). Linking groups that can be an alternative to the traditional amide-bond (“peptide bond”) linkages include, e.g., ketomethylene (e.g., —C(═O)—CH₂— for —C(═O)—NH—), aminomethylene (CH2-NH), ethylene, olefin (CH═CH), ether (CH₂—O), thioether (CH2-S), tetrazole (CN₄), thiazole, retroamide, thioamide, or ester (see, e.g., Spatola, Chemistry and Biochemistry of Amino Acids, Peptides and Proteins, Vol. 7, pp 267-357, “Peptide Backbone Modifications,” Marcell Dekker, NY (1983)) (157). A polypeptide can also be characterized as a mimetic by containing all or some non-natural residues in place of naturally occurring amino acid residues; non-natural residues are well described in the scientific and patent literature.

A “label” or a “detectable moiety” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins which can be made detectable, e.g. by incorporating a radiolabel into the peptide or used to detect antibodies specifically reactive with the peptide.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions.

An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” also encompass the ligation of nucleic acids having various coding regions or domains or promoter sequences from different sources into an expression cassette or vector for expression of, e.g., inducible or constitutive expression of a fusion protein comprising a translocation domain of the invention and a nucleic acid sequence amplified using a primer of the invention.

As used herein, a “stable cell line” refers to a cell line, which stably, i.e. over a prolonged period, expresses a heterologous nucleic sequence, i.e., a T1R, T2R or G protein. In preferred embodiments, such stable cell lines will be produced by transfecting appropriate cells, typically mammalian cells, e.g. HEK-293 cells, with a linearized vector that contains a T1R or T2R expression construct that expresses at least one T1R or T2R, i.e., T1R1, T1R2 and/or T1R3 or a T2R. Most preferably, such stable cell lines that express a functional T1R or T2R receptor will be produced by co-transfecting two linearized plasmids that express hT1R1 and hT1R3 or hT1R2 and hT1R3 or a single line plasmid that expresses a specific T2R and an appropriate selection procedure to generate cell lines having these genes stably integrated therein. Most preferably, the cell line will also stably express a G protein preferably a Gᵢ such as G_{αi} or G_{α15}.

“Antibody” refers to a polypeptide comprising a framework region from an immunoglobulin gene or fragment thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable light chain” (VL) and “variable heavy chain” (VH) refer to these light and heavy chains respectively.

A “chimeric antibody” is an antibody molecule in which (a) the constant region, or a portion then of, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

An “anti-T1R” antibody is an antibody or antibody fragment that specifically binds a polypeptide encoded by a T1R gene, cDNA, or a subsequence or variant thereof.

An “anti-T2R” antibody is an antibody or antibody fragment that specifically binds a polypeptide encoded by T2R gene, cDNA, or a subsequence or variant thereof.

An “anti-activated MAPK antibody” or an “anti-phospho MAPK antibody” refers to an antibody or antibody fragment that specifically binds to an activated (phosphorylated) form of MAPK

A “ligand that detects cAMP” is any moiety that specifically detects cAMP levels.

The term “immunoassay” is an assay that uses an antibody to specifically bind an antigen. The immunoassay is characterized by the use of specific binding properties of a particular antibody to isolate, target, and/or quantify the antigen. In a preferred embodiment of the invention, MAPK activity or cAMP levels will be immunoassayed in eukaryotic cells using an antibody that specifically recognizes an activated form of MAPK or cAMP.

The phrase “specifically (or selectively) binds” to an antibody or, “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a T1R or T2R family member from specific species such as rat, mouse, or human can be selected to obtain only those polyclonal antibodies that are specifically immunoreactive with the T1R or T2R polypeptide or an immunogenic portion thereof and not with other proteins, except for orthologs or polymorphic variants and alleles of the T1R or T2R polypeptide. This selection may be achieved by subtracting out antibodies that cross-react with T1R or T2R molecules from other species or other T1R or T2R molecules. Antibodies can also be selected that recognize only T1R GPCR family members but not GPCRs from other families. In the case of antibodies to activated MAPKs, suitable polyclonal and monoclonal antibodies are commercially available.

A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual, (1988) (59), for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “selectively hybridize” with another as defined above, or the ability of an antibody to “selectively (or specifically) bind to a protein, as defined above.

The term “expression vector” refers to any recombinant expression system for the purpose of expressing a nucleic acid sequence of the invention in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression “cassettes which contain only the minimum elements needed for transcription of the recombinant nucleic acid.

By “host cell” is meant a cell that contains an expression vector and supports the replication or expression of the expression vector. Host cells may be prokaryotic cells such as E. coli, or eukaryotic cells such as yeast, insect, amphibian, worm or mammalian cells such as CHO, Hela, BHK, HEK-293, and the like, e.g., cultured-cells, explants, and cells in vivo.

The terms “a,” “an,” and “the” are used in accordance with long-standing convention to refer to one or more.

The term “about”, as used herein when referring to a measurable value such as a percentage of sequence identity (e.g., when comparing nucleotide and amino acid sequences as described herein below), a nucleotide or protein length, an amount of binding, etc. is meant to encompass variations of ±20% or ±10%, more preferably ±5%, even more preferably ±1, and still more preferably ±1% from the specified amount, as such variations are appropriate to perform a disclosed method or otherwise carry out the present invention.

The term “substantially identical”, is used herein to describe a degree of similarity between nucleotide sequences, and refers to two or more sequences that have at least about least 60%, preferably at least about 70%, more preferably at least about 80%, more preferably about 90% to 99%, still more preferably about 95% to about 99%, and most preferably about 99% nucleotide identify, when compared and aligned for maximum correspondence, as measured using one of the following sequence comparison algorithms or by visual inspection. Preferably, the substantial identity exists in nucleotide sequences of at least about 100 residues, more preferably in nucleotide sequences of at least about 150 residues, and most preferably in nucleotide sequences comprising a full length coding sequence. The term “full length” is used herein to refer to a complete open reading frame encoding a functional T1R or T2R polypeptide, as described further herein below. Methods for determining percent identity between two polypeptides are defined herein below under the heading “Nucleotide and Amino Acid Sequence Comparisons”.

In one aspect, substantially identical sequences can be polymorphic sequences. The term “polymorphic” refers to the occurrence of two or more genetically determined alternative sequences or alleles in a population. An allelic difference can be as small as one base pair.

In another aspect, substantially identical sequences can comprise mutagenized sequences, including sequences comprising silent mutations. A mutation can comprise one or more residue changes, a deletion of residues, or all insertion of additional residues.

Another indication that two nucleotide sequences are substantially identical is that the two molecules hybridize specifically to or hybridize substantially to each other under stringent conditions. In the context of nucleic acid hybridization, two nucleic acid sequences being compared can be designated a “probe” and a “target.” A “probe” is a reference nucleic acid molecule, and a “target” is a test nucleic acid molecule, often found within a heterogeneous population of nucleic acid molecules. A “target sequence” is synonymous with a “test sequence.”

A preferred nucleotide sequence employed for hybridization studies or assays includes probe sequences that are complementary to or mimic at least an about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of the particular T1R or T2R. Such fragments can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” and “stringent hybridization wash conditions” refer to conditions under which a probe will hybridize to its target subsequence, typically in a complex mixture of nucleic acids but to no other sequences. Stringent conditions are sequence-dependent and will be different in different circumstances. Longer sequences hybridize specifically at higher temperatures. An extensive guide to the hybridization of nucleic acids is that in Tigssen; Techniques in Biochemistry and Molecular Biology—Hybridization With Nucleic Probes, “Overview of principles of hybridization and the strategy of nucleic acid assays.” (1973). Generally, highly stringent hybridization and wash conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium).

Stringent conditions will be those in which the salt concentration is less than about 1.0M sodium ion, typically about 0.01 to 1.0M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the additional of destabilizing agents such as formamide. For selective or specific hybridization, a positive signal is at least two times background, optionally 10 times background hybridization. Exemplary stringent hybridization conditions are:

50% formamide, 5×SSC, and 1% SDS, incubating at 42° C. or 5×SSC, 1% SDS, incubating at 65° C. The hybridization and wash steps effected in said exemplary stringent hybridization conditions are each effected for at least 1, 2, 5, 10, 15, 30, 60, or more minutes. Preferably, the wash and hybridization steps are each effected for at least 5 minutes, and more preferably, 10 minutes, 15 minutes, or more than 15 minutes.

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., eds (1989) Molecular Cloning: A Laboratory Manual, Cold Spring Harbor Laboratory Press, Cold Spring Harbor, N.Y. (60) for a description of SSC buffer. Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4× to 6×SSC at 40° C. For short probes (e.g., about 10 to 50 nucleotides), stringent conditions typically involve salt concentrations of less than about 1 M Na⁺ ion, typically about 0.01 to 1 M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are additional examples of hybridization and wash conditions that can be used to identify nucleotide sequences that are substantially identical to reference nucleotide sequences of the present invention: a probe nucleotide sequence preferably hybridizes to a target nucleotide sequence in 7% sodium dodecyl sulphate (SDS), 0.5M NaP0₄, 1 mM EDTA at 50° C. followed by washing in 2×SSC, 0.1% SDS at 50° C.; more preferably, a probe and target sequence hybridize in 7% sodium dodecyl sulphate (SDS), 0.5M NaP0₄, 1 mM EDTA at 50° C. followed by washing in 1×SSC, 0.1% SDS at 50° C.; more preferably, a probe and target sequence hybridize in 7% sodium dodecyl sulphate (SDS), 0.5M NaP04, 1 MM EDTA at 50° C. followed by washing in 0.5×SSC, 0.1% SDS at 50° C.; more preferably, a probe and target sequence hybridize in 7% sodium dodecyl sulphate, (SIDS), 0.5M NaP0₄, 1 mM EDTA at 50° C. followed by washing in 0.1×SSC, 0.1 SDS at 50° C.; more preferably, a probe and target sequence hybridize in 7% sodium dodecyl sulphate (SDS), 0.5M NaP0₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. Nucleic acid molecules that do not hybridize to each other under stringent conditions are still substantially identical if the corresponding proteins are substantially identical. This can occur, for example, when two nucleotide sequences comprise conservatively substituted variants as permitted by the genetic code.

Nucleic acids that do not hybridize to each other under stringent conditions are still substantially related if the polypeptides that they encode are substantially related. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such hybridizations and wash steps can be carried out for, e.g., 1, 2, 5, 10, 15, 30, 60, or more minutes. Preferably, the wash and hybridization steps are each effected for at least 5 minutes. A positive hybridization is at least twice background. Those of ordinary skill will readily recognize that alternative hybridization and wash conditions can be utilized to provide conditions of similar stringency.

The term “conservatively substituted variants” refers to nucleic acid sequences having degenerate codon substitutions wherein the third position of one or more selected (or all) codons is substituted with mixed-base and/or deoxyinosine residues. See Batzer et al. (1991) Nucleic Acids Res 19:5081; Ohtsuka et al. (1985) J Biol Chem 260:2605-2608; and Rossolini et al. (1994) Mol Cell Probes 8:91-98 (54-56).

The term T1R or T2R also encompasses nucleic acids comprising subsequences and elongated sequences of a T1R or T2R nucleic acid, including nucleic acids complementary to a T1R or T2R nucleic acid, T1R or T2R RNA molecules, and nucleic acids complementary to T1R or T2R RNAs (cRNAs).

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences,” as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison methods set forth below, or is defined as being capable of hybridizing to the nucleic acid segment in question under relatively stringent conditions such as those described herein. A particular example of a complementary nucleic acid segment is an antisense oligonucleotide.

The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted, sequence information, and recombinant derivation of an existing sequence.

The term “chimeric gene,” as used herein, refers to a promoter region operatively linked to a T1R or T2R sequence, including a T1R or T2R cDNA, a T1R or T2R nucleic acid encoding an antisense RNA molecule, a T1R or T2R nucleic acid encoding an RNA molecule having tertiary structure (e.g., a hairpin structure) or a T1R or T2R nucleic acid encoding a double-stranded RNA molecule. The term “chimeric gene” also refers to a T1R or T2R′ promoter region operatively linked to a heterologous sequence.

The term “operatively linked”, as used herein, refers to a functional combination between a promoter region and a nucleotide sequence such that the transcription of the nucleotide sequence is controlled and regulated by the promoter region. Techniques for operatively linking a promoter region to a nucleotide sequence are known in the art.

The term “vector” is used herein to refer to a nucleic acid molecule having nucleotide sequences that enable its replication in a host cell. A vector can also include nucleotide sequences to permit ligation of nucleotide sequences within the vector, wherein such nucleotide sequences are also replicated in a host cell. Representative vectors include plasmids, cosmids, and viral vectors. A vector can also mediate recombinant production of a T1R or T2R polypeptide, as described further herein below.

The term “construct”, as used herein to describe a type of construct comprising an expression construct, refers to a vector further comprising a nucleotide sequence operatively inserted with the vector, such that the nucleotide sequence is recombinantly expressed.

The terms “recombinantly expressed” or “recombinantly produced” are used interchangeably to refer generally to the process by which a polypeptide encoded by a recombinant nucleic acid is produced.

The term “heterologous nucleic acids” refers to a sequence that originates from a source foreign to an intended host cell or, if from the same source, is modified from its original form. Thus, preferably recombinant T1R or T2R nucleic acids comprise heterologous nucleic acids. A heterologous nucleic acid in a host cell can comprise a nucleic acid that is endogenous to the particular host cell but has been modified, for example by mutagenesis or by isolation from native cis-regulatory sequences. A heterologous nucleic acid also includes non-naturally occurring multiple copies of a native nucleotide sequence. A heterologous nucleic acid can also comprise a nucleic acid that is incorporated into a host cell's nucleic acids at a position wherein such nucleic acids are not ordinarily found.

Nucleic acids used in the cell-based assays of the present invention preferably MASK and cAMP assays can be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions are also known in the art. See e.g., Sambrook et al. (eds.) Molecular Cloning: A Laboratory Manual. Cold Spring Harbor Laboratory Press, Cold Spring Harbor, N.Y. (1989); Silhavy et al. Experiments with Gene Fusions. Cold Spring Harbor Laboratory Press, Cold Spring Harbor, N.Y. (1984); Glover & Hames DNA Cloning: A Practical Approach, 2nd ed. IRL Press and Oxford University Press, Oxford/New York (1995); Ausubel (ed.) Short Protocols in Molecular Biology, 3rd ed. Wiley, New York (1995) (60-68).

The term “substantially identical”, as used herein to describe a level of similarity between a particular T1R or T2R protein and a protein substantially identical to the T1R or T2R protein, refers to a sequence that is at least about 35% identical to the particular T1R or T2R protein, when compared over the full length of the T1R or T2R protein. Preferably, a protein substantially identical to the T1R or T2R protein used in the present invention comprises an amino acid sequence that is at least about 35% to about 45% identical to a particular T1R or T2R, more preferably at least about 45% to about 55% identical thereto, even more preferably at least about 55% to about 65% identical thereto, still more preferably at least about 65% to about 75% identical thereto, still more preferably at least about 75% to about 85% identical thereto, still more preferably at least about 85% to about 95% identical thereto, and still more preferably at least about 95% to about 99% identical thereto when compared over the full length of the particular T1R or T2R. The term “full length” refers to a functional T1R or T2R polypeptide. Methods for determining percent identity between two polypeptides are also defined herein below under the heading “Nucleotide and Amino Acid Sequence Comparisons”.

The term “substantially identical,” when used to describe polypeptides, also encompasses two or more polypeptides sharing a conserved three-dimensional structure. Computational methods can be used to compare structural representations, and structural models can be generated and easily tuned to identify similarities around important active sites or ligand binding sites. See Saqi et al. Bioinformatics 15:521-522 (1999); Barton Acta Crystallogr D Biol Crystallogr 54:1139-1146 (1998); Henikoff et al. Electrophoresis 21:1700-1706 (2000); and Huang et al. Pac Symp Biocomput: 230-241 (2000) (64-67).

Substantially identical proteins also include proteins comprising amino acids that are functionally equivalent to a T1R or T2R according to the invention. The term “functionally equivalent” in the context of amino acids is known in the art and is based on the relative similarity of the amino acid side-chain substituents. See Henikoff & Henikoff Adv Protein Chem 54:73-97 (2000) (68). Relevant factors for consideration include side-chain hydrophobicity, hydrophilicity, charge, and size. For example, arginine, lysine, and histidine are all positively charged residues; that alanine, glycine, and serine are all of similar size; and that phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al., J. Mol. Biol. 157(1):105-32 (1982)) (69). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those which are within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within ±1 of the original value are particularly preferred, and those within ±0.5 of the original value are even more particularly preferred.

The term “substantially identical” also encompasses polypeptides that are biologically functional equivalents of a particular T1R or T2R polypeptide. The term “functional” includes an activity of an T1R or T2R polypeptide, for example activating intracellular signaling pathways (e.g., coupling with gustducin) and mediating taste perception. Preferably, such activation shows a magnitude and kinetics that are substantially similar to that of a cognate T1R or T2R polypeptide in vivo. Representative methods for assessing T1R or T2R activity are described in the patent applications incorporated by reference, herein.

The assays of the present invention also can use functional fragments of a particular T1R or T2R polypeptide. Such functional portion need not comprise all or substantially all of the amino acid sequence of a native. T1R or T2R gene product. The assays of the present invention also can use functional polypeptide sequences that are longer sequences than that of a native T1R or T2R polypeptide. For example, one or more amino acids can be added to the N-terminus or C-terminus of a T1R or T2R polypeptide. Such additional amino acids can be employed in a variety of applications, including but not limited to purification applications. Methods of preparing elongated proteins are known in the art.

“MAPK” or “MAP Kinase” refers to a mitogen activated protein kinase, the expression of which is activated by some functional GPCRs, i.e., T2Rs and T1Rs.

“MAPK” or “MAP Kinase” activation specific ligands” refers to a ligand, preferably a polyclonal or monoclonal antibody or fragment thereof that specifically binds an activated form of MAPK, e.g., p42/p44 MAPK or p38/MAPK Antibodies that specifically bind the activated (phosphorylated) form of MAPK are commercially available and include the phosph-p44/p42 MAP Kinase antibody #9106 available from Cell Signaling Technologies, the polyclonal anti-phospho-p44/42 MAPK and anti-phospho-p38 MAPK antibodies available from UBI, (Lake Placid, N.Y., USA) and New England Biolabs (Beverly, Mass., USA), the anti-phospho-p44/42 MAPK antibodies reported by Discovery Research Laboratories III, Takeda Chemical Indust. Ltd., (Oskaka Japan) (Tan et al., J. Immunol. Meth. 232(1-2): 87-97 (1998)) (70).

“Ligand” or “compound” that “activates MAPK” refers to a compound which when contacted with a eukaryotic cell that expresses a functional GPCR, herein at least one functional T1R or T2R, results in a detectable increase in the activated form of MAPK This increase will preferably will be detected by antibody-based detection methods that use an antibody that specifically binds to an activated form of MAPK

“PLC” refers to phospholipase C. In the present invention, “a ligand or compound that activates MAPK” may activate MAPK in cells via a pathway that is independent of PLC activation.

### Cell Based Assays of the Present Invention

This, in one aspect, present invention generally relates to cell-based assays for identifying compounds that modulate the activity of at least one T1R or T2R taste receptor, wherein the assays comprise contacting a eukaryotic cell that stably or transiently expresses at least one functional T1R or T2R and a G protein that functionally couples therewith, e.g. a Gᵢ protein such as Gαᵢ with a putative modulator of said functional T1R or T2R, and assaying the effect of said putative agonist or antagonist compound on Gᵢ mediated signaling pathways, e.g., by assaying the effect of said putative modulation on MAPK activation, cAMP accumulation or adenylyl cyclase activity. For example, a modulator compound will result, e.g., in a detectable increase or decrease in the amount of an activated form of MAPS, i.e., phosphorylated MAPK, e.g., phosphorylated p44/42 MAP Kinase or phosphorylated p38 MAP Kinase, and will elicit this effect on MAPK activation by a pathway independent of PLC activation or will result in detectable increase or decrease in cAMP accumulation, or will result in a change (e.g., decrease) in adenylyl cyclase activity. However, the invention embraces any cell-based assays that identify compounds that modulate to a TRGPCR (T1R or T2R)/G_{αi} mediated signaling pathway.

The eukaryotic cells used in the subject assays, preferably MAPK, cAMP or adenylyl cyclase assays, will stably or transiently express at least one functional T1R or T2R. Preferably, the eukaryotic cell will either stably or transiently express a functional T1R1/T1R3 umami taste receptor or a functional T1R2/T1R3 sweet taste receptor or will stably or transiently express a desired functional T2R, preferably a functional human T1R or T2R taste receptor. In order to produce a functional taste receptor, the eukaryotic cell will further be transfected to stably or transiently express or will endogenously express a G protein that couples with said T1R(s) or T2R thereby resulting in a functional taste receptor. Examples of suitable G proteins are known in the art and are referred in the patent applications incorporated by reference herein. In a preferred embodiment, the G protein will comprise a Gi protein selected from Gαᵢ, i.e. Gαᵢ₁₋₁, Gαᵢ₁₋₂, Gαᵢ₁₋₃, Gαᵢ₀₋₁, and Gαᵢ₀₋₂. Alternatively, the G protein will comprise Gα₁₅, α-transcucin, gustducin, G_{αz} or a functional chimera or variant thereof that couples with the T1R(s) or T2R expressed by the eukaryotic cell.

The present assays can be effected using any eukaryotic cell that functionally expresses the particular T1R(s) or T2R, and which cell, when contacted with an activator of said T1R or T1R results in an increase in an activated form of MAPK, or a decrease in cAMP accumulation or a reduction in adenylyl cyclase activity by a pathway that is independent of PLC activation. Examples of suitable eukaryotic cells include amphibian, yeast, insect, amphibian, worm and mammalian cells. Specific examples of suitable cells for use in the subject cell-based assays include HEK293 cells, BHK cells, CHO cells, Hela cells and Xenopus oocytes.

In a preferred embodiment the eukaryotic cells used in the subject cell-based assays, e.g., MAPK, cAMP and adenylyl cyclase assays will comprise HEK293 cells that stably or transiently express at least one or functional T1R or T2R taste receptor by the transfection of such cells with a cDNA or cDNAs encoding said at least one T1R or T2R. For example, HEK293 cells stably expressing the large T cell antigen and the promiscuous G protein Gα₁₅ (HEK293T-Gα₁₅) or G_{αi} can be transiently transfected with a particular taste receptor plasmid by known transfection methods, e.g., by use of Ca²⁺ phosphate or lipid-based systems, or other transformation methods referenced supra. As noted previously, the T1R or T2R expressing cell will further express endogenously or be engineered to express a G protein that functionally couples therewith, e.g., a G protein selected from the G_{αi} proteins identified previously.

Cells that stably or transiently express the particular taste receptor are used in assays that measure the effect of at least one putative T1R or T2R modulatory compound on G_{αi}-mediated signaling pathways, e.g., by measuring its effect on MAPK activation, cAMP accumulation or adenylyl cyclase activity. The MAPK or cAMP assays of the present invention can use immobilized cells or cells in suspension. In a preferred embodiment the taste receptor expressing cells will be seeded into multi-well culture plates, e.g., 6-well culture plates. However, other in vitro cell culture devices can be substituted therefore, and is not critical to the invention.

In a typical MAPK or cAMP assay according to the invention, functional expression of the T1R or T2R expressing eukaryotic cell is allowed to proceed for a certain time, e.g., on the order of about 48 hours, and then taste receptor expressing cells are stimulated with a putative modulatory compound for a fixed time, e.g., about 5 minutes, and then the reaction is then stopped, e.g., by the addition of ice-cold buffer, and the cells are then, assayed for changes in activated MAPK, cAMP or adenylyl cyclase activity. However, these reaction times may be shortened or lengthened within wide limits.

The level of activated MAPK produced by such cells is detected in whole cells or cell lysates. In a preferred embodiment, cell lysates are prepared by known methods, and detected by activated cAMP, MAPK or adenylyl cyclase activity is detected by known methods. For example, activated MAPK can be the use of a polyclonal or monoclonal antibody or fragment thereof that specifically recognizes an activated (phosphorylated) form of MAPK In a preferred embodiment, activation of MAPK is detected by Western analysis of cell lysates using a specific monoclonal antibody that recognizes phosphorylated (active) MAPK (Phospho-p44/42 MAP Kinase antibody #9106 available from Cell Signaling Technologies) or another commercially available antibody that specifically recognizes activated MAPK

### Exemplification of Cell-Based Assays According to the Invention

The following are exemplary of cell-based assays that may be used according to the invention for detecting the effect of a putative modulator on T1R or T2R activity.

### 1. GTP Assay

For GPCRs T1R OR T2R, a measure of receptor activity is the binding of GTP by cell membranes containing receptors. In the method described by Traynor and Nahorski, 1995, Mol. Pharmacol. 47: 848-854, (1995) (130) one essentially measures G-protein coupling to membranes by detecting the binding of labelled GTP. For GTP binding assays, membranes isolated from cells expressing the receptor are incubated in a buffer containing 20 mM HEPES, pH 7.4, 100 mM NaCl, and 10 mM MgCl₂, 80 pM .³⁵S-GTPγS and 3 μM GDP.

The assay mixture is incubated for 60 minutes at 30° C., after which unbound labelled GTP is removed by filtration onto GF/B filters. Bound, labelled GTP is measured by liquid scintillation counting. The presence and absence of a candidate modulator of T1R or T2R activity. A decrease of 10% or more in labelled GTP binding as measured by scintillation counting in an assay of this kind containing a candidate modulator, relative to an assay without the modulator, indicates that the candidate modulator inhibits T1R or T2R activity. A compound is considered an agonist if it induces at least 50% of the level of GTP binding when the compound is present at 1 μM or less.

GTPase activity is measured by incubating the membranes containing a T1R or T2R polypeptide with .γ³²P-GTP. Active GTPase will release the label as inorganic phosphate, which is detected by separation of free inorganic phosphate in a 5% suspension of activated charcoal in 20 mM H₃PO₄, followed by scintillation counting. Controls include assays using membranes isolated from cells not expressing T1R or T2R (mock-transfected), in order to exclude possible non-specific effects of the candidate compound.

In order to assay for the effect of a candidate modulator on T1R or T2R-regulated GTPase activity, membrane samples are incubated with and without the modulator, followed by the GTPase assay. A change (increase or decrease) of 10% or more in the level of GTP binding or GTPase activity relative to samples without modulator is indicative of T1R or T2R modulation by a candidate modulator.

### 2. Downstream Pathway Activation Assays

i) Calcium Flux—The Aequorin-based Assay:

The aequorin assay takes advantage of the responsiveness of mitochondrial apoaequorin to intracellular calcium release induced by the activation of GPCRs (Stables et al., Anal. Biochem. 252:115-126 (1997); Detheux et al., 2000, J. Exp. Med., 192 1501-1508 (2000) (131-132); both of which are incorporated herein by reference). Briefly, T1R or T2R-expressing clones are transfected to coexpress mitochondrial apoaequorin and G_{α16}. Cells are incubated with 5 μM Coelenterazine H (Molecular Probes) for 4 hours at room temperature, washed in DMEM-F12 culture medium and resuspended at a concentration of 0.5.times.10.sup.6 cells/ml. Cells are then mixed with test agonist molecules and light emission by the aequorin is recorded with a luminometer for 30 seconds. Results are expressed as Relative Light Units (RLU). Controls include assays using membranes isolated from cells not expressing T1R or T2R (mock transfected), in order to exclude possible non-specific effects of the candidate compound.

Aequorin activity or intracellular calcium levels are “changed” if light intensity increases or decreases by 10% or more in a sample of cells, expressing a T1R or T2R polypeptide and treated with a candidate modulator, relative to a sample of cells expressing the T1R or T2R polypeptide but not treated with the candidate modulator or relative to a sample of cells not expressing the T1R or T2R polypeptide (mock-transfected cells) but treated with the candidate modulator.

ii) Adenylate Cyclase Assay:

Assays for adenylate cyclase activity are described by Kenimer & Nirenberg, Mol. Pharmacol. 20: 585-591 (1981) (133). That assay is a modification of the assay taught by Solomon et al., 1974, Anal. Biochem. 58: 541-548 (1974) (134), also incorporated herein by reference. Briefly, 100 μl reactions contain 50 mM Tris-Hcl (pH 7.5), 5 mM MgCl₂, 20 mM creatine phosphate (disodium salt), 10 units (71 μg of protein) of creatine phosphokinase, 1 mM α-_{32P} (tetrasodium salt, 2 μCᵢ), 0.5 mM cyclic AMP, G-^{3H}-labeled cyclic AMP (approximately 10,000 cpm), 0.5 mM Ro20-1724, 0.25% ethanol, and 50-200 μg of protein homogenate to be tested (i.e., homogenate from cells expressing or not expressing a T1R or T2R polypeptide, treated or not treated with a candidate modulator). Reaction mixtures are generally incubated at 37° C. for 6 minutes. Following incubation, reaction mixtures are deproteinized by the addition of 0.9 ml of cold 6% trichloroacetic acid. Tubes are centrifuged at 1800×g for 20 minutes and each supernatant solution is added to a Dowex AG50W-X4 column. The cAMP fraction from the column is eluted with 4 ml of 0.1 mM imidazole-HCl (pH 7.5) into a counting vial. Assays should be performed in triplicate. Control reactions should also be performed using protein homogenate from cells that do not express a T1R or T2R polypeptide.

According to the invention, adenylate cyclase activity is “changed” if it increases or decreases by 10% or more in a sample taken from cells treated with a candidate modulator of T1R or T2R activity, relative to a similar sample of cells not treated with the candidate modulator or relative to a sample of cells not expressing the T1R or T2R polypeptide (mock-transfected cells) but treated with the candidate modulator.

iii) cAMP Assay:

Intracellular or extracellular cAMP is measured using a cAMP radioimmunoassay (RIA) or cAMP binding protein according to methods widely known in the art. For example, Horton & Baxendale, Methods Mol. Biol. 41: 91-105 (1995) (135), which is incorporated herein by reference, describes an RIA for cAMP.

A number of kits for the measurement of cAMP are commercially available, such as the High Efficiency Fluorescence Polarization-based homogeneous assay marketed by LJL Biosystems and NEN Life Science Products. Control reactions should be performed using extracts of mock-transfected cells to exclude possible non-specific effects of some candidate modulators.

The level of cAMP is “changed” if the level of cAMP detected in cells, expressing a T1R or T2R polypeptide and treated with a candidate modulator of T1R or T2R activity (or in extracts of such cells), using the RIA-based assay of Horton & Baxendale, 1995 (135), increases or decreases by at least 10% relative to the cAMP level in similar cells not treated with the candidate modulator.

(iv) Phospholipid Breakdown, DAG Production and Inositol Triphosphate Levels:

Receptors that activate the breakdown of phospholipids can be monitored for changes due to the activity of known or suspected modulators of T1R or T2R by monitoring phospholipid breakdown, and the resulting production of second messengers DAG and/or inositol triphosphate (IP₃). Methods of detecting each of these are described in Phospholipid Signalling Protocols, edited by Ian M. Bird. Totowa, N.J., Humana Press, (1998) (136), which is incorporated herein by reference. See also Rudolph et al., J. Biol. Chem. 274: 11824-11831 (1999) (137), which also describes an assay for phosphatidylinositol breakdown. Assays should be performed using cells or extracts of cells expressing T1R or T2R, treated or not treated or without a candidate modulator. Control reactions should be performed using mock-transfected cells or extracts from them in order to exclude possible non-specific effects of some candidate modulators.

According to the invention, phosphatidylinositol breakdown, and diacylglycerol and/or inositol triphosphate levels are “changed” if they increase or decrease by at least 10% in a sample from cells expressing a T1R or T2R polypeptide and treated with a candidate modulator, relative to the level observed in a sample from cells expressing a T1R or T2R polypeptide that is not treated with the candidate modulator.

(v) PKC Activation Assays:

Growth factor receptor tyrosine kinases can signal via a pathway involving activation of Protein Kinase C (PKC), which is a family of phospholipid- and calcium-activated protein kinases. PKC activation ultimately results in the transcription of an array of proto-oncogene transcription factor-encoding genes, including c-fos, c-myc and c-jun, proteases, protease inhibitors, including collagenase type I and plasminogen activator inhibitor, and adhesion molecules, including intracellular adhesion molecule I (ICAM I). Assays designed to detect increases in gene products induced by PKC can be used to monitor PKC activation and thereby receptor activity. In addition, the activity of receptors that signal via PKC can be monitored through the use of reporter gene constructs driven by the control sequences of genes activated by PKC activation. This type of reporter gene-based assay is discussed in more detail below.

For a more direct measure of PKC activity, the method of Kikkawa et al., 1982, J. Biol. Chem. 257: 13341 (1982) (138), can be used. This assay measures phosphorylation of a PKC substrate peptide, which is subsequently separated by binding to phosphocellulose paper. This PKC assay system can be used to measure activity of purified kinase, or the activity in crude cellular extracts. Protein kinase C sample can be diluted in 20 mM HEPES/2′ mM DTT immediately prior to assay.

The substrate for the assay is the peptide Ac-FKKSFKL-NH₂, derived from the myristoylated alanine-rich protein kinase C substrate protein (MARCKS). The Kₘ of the enzyme for this peptide is approximately 50 μM. Other basic, protein kinase C-selective peptides known in the art can also be used, at a concentration of at least 2-3 times their Kₘ. Cofactors required for the assay include calcium, magnesium, ATP, phosphatidylserine and diacylglycerol. Depending upon the intent of the user, the assay can be performed to determine the amount of PKC present (activating conditions) or the amount of active PKC present (non-activating conditions). For most purposes according to the invention, non-activating conditions will be used, such that the PKC, that is active in the sample when it is isolated, is measured, rather than measuring the PKC that can be activated. For non-activating conditions, calcium is omitted from the assay in favor of EGTA.

The assay is performed in a mixture containing 20 mM HEPES, pH 7.4, 1-2 mM DTT, 5 mM MgCl₂, 100 μM ATP, .about. 1 μCᵢ .γ³²P-ATP, 100 μg/ml peptide substrate (⁻100 μM), 140 μM/3.8 μM phosphatidylserine/diacylglycerol membranes, and 100 μM calcium (or 500 μM EGTA). 48 μL of sample, diluted in 20 mM HEPES, pH 7.4, 2 mM DTT is used in a final reaction volume of 80 μl. Reactions are performed at 30° C. for 5-10 minutes, followed by addition of 25 μl of 100 mM ATP, 100 mM EDTA, pH 8.0, which stops the reactions.

After the reaction is stopped, a portion (85 μl) of each reaction is spotted onto a Whatman P81 cellulose phosphate filter, followed by washes: four times 500 ml in 0.4% phosphoric acid, (5-10 min per wash); and a final wash in 500 ml 95% EtOH, for 2-5 min. Bound radioactivity is measured by scintillation counting. Specific activity (cpm/nmol) of the labelled ATP is determined by spotting a sample of the reaction onto PS1 paper and counting without washing. Units of PKC activity, defined as nmol phosphate transferred per min, are then calculated by known methods.

An alternative assay can be performed using a Protein Kinase C Assay Kit sold by PanVera (Cat. # P2747).

Assays are performed on extracts from cells expressing a T1R or T2R polypeptide, treated or not treated with a candidate modulator. Control reactions should be performed using mock-transfected cells, or extracts from them in order to exclude possible non-specific effects of some candidate modulators.

According to the invention, PKC activity is “changed” by a candidate modulator when the units of PKC measured by either assay described above increase or decrease by at least 10%, in extracts from cells expressing T1R or T2R and treated with a candidate modulator, relative to a reaction performed on a similar sample from cells not treated with a candidate modulator.

(iv) Kinase Assays:

MAP Kinase assays have already been described supra. MAP kinase activity can be assayed using any of several kits available commercially, for example, the p38 MAP Kinase assay kit sold by New England Biolabs (Cat #-9820) or the FlashPlate™ MAP Kinase assays sold by Perkin-Elmer Life Sciences.

MAP Kinase activity is “changed” if the level of activity is increased or decreased by 10% or more in a sample from cells, expressing a T1R or T2R polypeptide, treated with a candidate modulator relative to MAP kinase activity in a sample from similar cells not treated with the candidate modulator.

Direct assays for tyrosine kinase activity using known synthetic or natural tyrosine kinase substrates and labelled phosphate are well known, as are similar assays for other types of kinases (e.g., Ser/Thr kinases). Kinase assays can be performed with both purified kinases and crude extracts prepared from cells expressing a T1R or T2R polypeptide, treated with or without a candidate modulator. Control reactions should be performed using mock-transfected cells, or extracts from them in order to exclude possible non-specific effects of some candidate modulators. Substrates can be either full-length protein or synthetic peptides representing the substrate. Pinna & Ruzzene (Biochem. Biophys. Acta 1314: 191-225 (1996) (139)) list a number of phosphorylation substrate sites useful for detecting kinase activities. A number of kinase substrate peptides are commercially available. One that is particularly useful is the “Src-related peptide,” RRLIEDAEYAARG (available from Sigma # A7433), which is a substrate for many receptor and nonreceptor tyrosine kinases. Because the assay described below requires binding of peptide substrates to filters, the peptide substrates should have a net positive charge to facilitate binding. Generally, peptide substrates should have at least 2 basic residues and a free amino terminus. Reactions generally use a peptide concentration of 0.7-1.5 mM.

Assays are generally carried out in a 25 μl volume comprising 5 .mu.l of 5× kinase buffer (5 mg/mL BSA, 150 mM Tris-Cl (pH 7.5), 100 mM MgCl₂; depending upon the exact kinase assayed for, in Cl₂ can be used in place of or in addition to the MgCl₂), 5 .mu.l of 1.0 mM ATP (0.2 mM final concentration), γ³²P-ATP (100-500 cpm/pmol), 3 μl of 10 mM peptide substrate (1.2 mM final concentration), cell extract containing kinase to be tested (cell extracts used for kinase assays should contain a phosphatase inhibitor (e.g. 0.1-1 mM sodium orthovanadate)), and H₂O to 25 μl. Reactions are performed at 30° C., and are initiated by the addition of the cell extract.

Kinase reactions are performed for 30 seconds to about 30 minutes, followed by the addition of 45 μl of ice-cold 10% trichloroacetic acid (TCA). Samples are spun for 2 minutes in a microcentrifuge, and 35 μl of the supernatant is spotted onto Whatman P81 cellulose phosphate filter circles. The filters are washed three times with 500 ml cold 0.5% phosphoric acid, followed by one wash with 200 ml of acetone at room temperature for 5 minutes. Filters are dried and incorporated ³²P is measured by scintillation counting. The specific activity, of ATP in the kinase reaction (e.g., in cpm/pmol) is determined by spotting a small sample (2-5 μl) of the reaction onto a P81 filter circle and counting directly, without washing. Counts per minute obtained in the kinase reaction (minus blank) are then divided by the specific activity to determine the moles of phosphate transferred in the reaction.

Tyrosine kinase activity is “changed” if the level of kinase activity is increased or decreased by 10% or more in a sample from cells, expressing a T1R or T2R polypeptide, treated with a candidate modulator relative to kinase activity in a sample from similar cells not treated with the candidate modulator.

(vii) Transcriptional Reporters for Downstream Pathway Activation:

The intracellular signal initiated by binding of an agonist to a receptor, e.g., T1R or T2R, sets in motion a cascade of intracellular events, the ultimate consequence of which is a rapid and detectable change in the transcription or translation of one or more genes. The activity of the receptor can therefore be monitored by detecting the expression of a reporter gene driven by control sequences responsive to T1R or T2R activation.

As used herein “promoter” refers to the transcriptional control elements necessary for receptor-mediated regulation of gene expression, including not only the basal promoter, but also any enhancers or transcription-factor binding sites necessary for receptor-regulated expression. By selecting promoters that are responsive to the intracellular signals resulting from agonist binding, and operatively linking the selected promoters to reporter genes whose transcription, translation or ultimate activity is readily detectable and measurable, the transcription based reporter assay provides a rapid indication of whether a given receptor is activated.

Reporter genes such as luciferase, CAT, GFP, β-lactamase or β-galactosidase are well, known in the art, as are assays for the detection of their products.

Genes particularly well suited for monitoring receptor activity are the “immediate early” genes, which are rapidly induced, generally within minutes of contact between the receptor and the effector protein or ligand. The induction of immediate early gene transcription does not require the synthesis of new regulatory proteins. In addition to rapid responsiveness to ligand binding, characteristics of preferred genes useful for making reporter constructs include: low or undetectable expression in quiescent cells; induction that is transient and independent of new protein synthesis; subsequent shut-off of transcription requires new protein synthesis; and mRNAs transcribed from these genes have a short half-life. It is preferred, but not necessary that a transcriptional control element have all of these properties for it to be useful.

An example of a gene that is responsive to a number of different stimuli is the c-fos proto-oncogene. The c-fos gene is activated in a protein-synthesis-independent manner by growth factors, hormones, differentiation-specific agents, stress, and other known inducers of cell surface proteins. The induction of c-fos expression is extremely rapid, often occurring within minutes of receptor stimulation. This characteristic makes the c-fos regulatory regions particularly attractive for use as a reporter of receptor activation.

The c-fos regulatory elements include (see, Verma et al., Cell 51: 513-514) (1987) (140): a TATA box that is required for transcription initiation; two upstream elements for basal transcription, and an enhancer, which includes an element with dyad symmetry and which is required for induction by TPA, serum, EGF, and PMA.

The 20 bp c-fos transcriptional enhancer element located between −317 and −298 bp upstream from the c-fos mRNA cap site, is essential for serum induction in serum starved NIH 3T3 cells. One of the two upstream elements is located at −63 to −57 and it resembles the consensus sequence for cAMP regulation.

The transcription factor CREB (cyclic AMP responsive element binding protein) is, as the name implies, responsive to levels of intracellular cAMP. Therefore, the activation of a receptor that signals via modulation of cAMP levels can be monitored by detecting either the binding of the transcription factor, or the expression of a reporter gene linked to a CREB-binding element (termed the CRE, or cAMP response element). The DNA sequence of the CRE is TGACGTCA. (Reporter constructs responsive to CREB binding activity are described in U.S. Pat. No. 5,919,649) (141).

Other promoters and transcriptional control elements, in addition to the c-fos elements and CREB-responsive constructs, include the vasoactive intestinal peptide (VIP) gene promoter (cAMP responsive; Fink et al., 1988, Proc. Natl. Acad. Sci. 85:6662-6666) (1988) (142); the somatostatin gene promoter (cAMP responsive; Montminy et al., Proc. Natl. Acad. Sci. 83:6682-6686 (1986) (143)); the proenkephalin promoter (responsive to cAMP, nicotinic agonists, and phorbol esters; Comb et al., Nature 323:353-356 (1986)-(144)); the phosphoenolpyruvate carboxy-kinase (PEPCK) gene promoter (cAMP responsive; Short et al., J. Biol. Chem. 261:9721-9726 (1986) (145)).

Additional examples of transcriptional control elements that are responsive to changes in GPCR activity include, but are not limited to those responsive to the AP-1 transcription factor and those responsive to NF-KB activity. The consensus AP-1 binding site is the palindrome TGA(C/G)TCA (Lee et al., Nature 325: 368-372 (1987) (146); Lee et al., Cell 49: 741-752 (1987) (147)). The AP-1 site is also responsible for mediating induction by tumor promoters such as the phorbol ester 12-O-tetradecanoylphorbol-.beta.-acetate (TPA), and are therefore sometimes also referred to as a TRE, for TPA-response element. AP-1 activates numerous genes that are involved in the early response of cells to growth stimuli. Examples of AP-1-responsive genes include, but are not limited to the genes for Fos and Jun (which proteins themselves make up AP-1-activity), Fos-related antigens (Fra) 1 and 2, I κβα, ornithine decarboxylase, and annexins I and II.

The NF-KB binding element has the consensus sequence GGGGACTTTCC. A large number of genes have been identified as NF-KB responsive, and their control elements can be linked to a reporter gene to monitor GPCR activity. A small sample of the genes responsive to NF-KB includes those encoding IL-1β. (Hiscott et al., Mol. Cell. Biol. 13:6231-6240 (1993) (148)), TNF-α (Shakhov et al., J. Exp. Med. 171: 35-47 (1990) (149)), CCR5 (Liu et al., AIDS Res. Hum. Retroviruses 14: 1509-1519 (1998) (150)), P-selectin (Pan, & McEver, J. Biol. Chem. 270: 23077-23083 (1995) (151)), Fas ligand (Matsui et al., J. Immunol. 161: 3469-3473 (1998) (152)), GM-CSF (Schreck & Baeuerle, Mol. Cell. Biol. 10: 1281-1286 (1990) (153)) and Iκβα (Haskill et al., Cell 65: 1281-1289 (1991) (154)). Vectors encoding NF-KB-responsive reporters are also known in the art or can be readily made by one of skill in the art using, for example, synthetic NF-KB elements and a minimal promoter, or using the NF-KB-responsive sequences of a gene known to be subject to NF-KB regulation. Further, NF-KB responsive reporter constructs are commercially available e.g. from CLONTECH.

To screen for agonists, the cells are left untreated, exposed to candidate modulators, and expression of the reporter is measured. An increase of at least 50% in reporter expression in the presence of a candidate modulator indicates that the candidate is a modulator of T1R or T2R activity. An agonist will induce at least as many, and preferably the same amount or more of reporter expression than buffer alone. This approach can also be used to screen for inverse agonists where cells express a T1R or T2R polypeptide at levels such that there is an elevated basal activity of the reporter. A decrease in reporter activity of 10% or more in the presence of a candidate modulator, relative to its absence, indicates that the compound is an inverse agonist.

To screen for antagonists, the cells expressing T1R or T2R and carrying the reporter construct are contacted in the presence and absence of a candidate modulator. A decrease of 10% or more in reporter expression in the presence of candidate modulator, relative to the absence of the candidate modulator, indicates that the candidate is a modulator of T1R or T2R activity.

Controls for transcription assays include cells not expressing T1R or T2R but carrying the reporter construct, as well as cells with a promoterless reporter construct. Compounds that are identified as modulators of T1R or T2R-regulated transcription should also be analyzed to determine whether they affect transcription driven by other regulatory sequences and by other receptors, in order to determine the specificity and spectrum of their activity.

The transcriptional reporter assay, and most cell-based assays, are well suited for screening expression libraries for proteins for those that modulate T1R or T2R activity. The libraries can be, for example, cDNA libraries from natural sources, e.g., plants, animals, bacteria, etc., or they can be libraries expressing randomly or systematically mutated variants of one or more polypeptides. Genomic libraries in viral vectors can also be used to express the mRNA content of one cell or tissue, in the different libraries used for screening of T1R or T2R.

(viii) Inositol Phosphate (IP) Measurement:

Cells of the invention are labelled for 24 hours with 10 μCi/ml³H] inositol in inositol free DMEM containing 5% FCS, antibiotics, amphotericin, sodium pyruvate and 400 μg/ml G418. Cells are incubated for 2 h in Krebs-Ringer Hepes (KRH) buffer of the following composition (124 mM NaCl, 5 mM KCl, 1.25 mM MgSO₄, 1.45 mM CaCl₂, 1.25 mM KH2PO₄, 25 mM Hepes (pH:7.4) and 8 mM glucose). The cells are then challenged with various nucleotides for 30 s. The incubation is stopped by the addition of an ice cold 3% perchloric acid solution. IP are extracted and separated on Dowex columns as previously described. 2MeSATP and ATP solutions (1 mM) are treated at room temperature with 20 units/ml CPK and 10 Mm cp for 90 min to circumvent problems arising from the contamination and degradation of triphosphate nucleotide solutions.

T1R or T2R Assay

The invention provides for an assay for detecting the activity of a receptor, of the invention in a sample. For example, T1R or T2R activity can be measured in a sample comprising a cell or a cell membrane that expresses T1R or T2R. The assay is performed by incubating the sample in the presence or absence of a modulator and carrying out a second messenger assay, as described above. The results of the second messenger assay performed in the presence or absence of the activator are compared to determine if the T1R or T2R receptor is active.

Any of the assays of receptor activity, including but not limited to the GTP-binding, GTPase, adenylate cyclase, cAMP, phospholipid-breakdown, diacylglycerol, inositol triphosphate, arachidonic acid release (see below), PKC, kinase and transcriptional reporter assays, can be used to determine the presence of an agent in a sample, e.g., a tissue sample, that affects the activity of the T1R or T2R receptor molecule. To do so, T1R or T2R polypeptide is assayed for activity in the presence and absence of the sample or an extract of the sample. An increase in T1R or T2R activity in the presence of the sample or extract relative to the absence of the sample indicates that the sample contains an agonist of the receptor activity. A decrease in receptor activity in the presence of an agonist and the sample, relative to receptor activity in the absence thereof, indicates that the sample contains an antagonist of T1R or T2R activity.

The amount of increase or decrease in measured activity necessary for a sample to be said to contain a modulator depends upon the type of assay used. Generally, a 10% or greater change (increase or decrease) relative to an assay performed in the absence of a sample indicates the presence of a modulator in the sample. One exception is the transcriptional reporter assay, in which at least a two-fold increase or 10% decrease in signal is necessary for a sample to be said to contain a modulator. It is preferred that an agonist stimulates at least 50%, and preferably 75% or 100% or more, e.g., 2-fold, 5-fold, 10-fold or greater receptor activation.

Other functional assays include, for example, microphysiometer or biosensor assays (see Hafner, 2000, Biosens. Bioelectron. 15: 149-158) (2000) (155)).

As described in detail infra, it has been found that cell-based assays according to the invention, e.g., MAPK and cAMP assay methods exemplified, enable the detection of robust activation of bitter taste receptors (mT2R05) and hT2R04 as well as the sweet receptor (T1R2/T1R3) and umami receptor (T1R1/T1R3). (These results are discussed in detail in the examples and the figures referred to therein.) It is anticipated further, based on these results, that cell-based assays that detect the effect of putative modulator on Gᵢ/T1R or Gᵢ/TR mediated signaling pathways, e.g., MAPK and cAMP assays, will be identify compounds that modulate the activity of any functional taste receptor comprising a T1R or T2R polypeptide or functional fragment.

Additionally, the results obtained indicate that the responses obtained are receptor-dependent and receptor-specific. For example, the parental cell lines HEK293 or HEK293T-G₁₅ do not exhibit comparable activation of MAPK or a reduction in cAMP (See FIGS. 1-7) when stimulated with the same agonists.

Further, it has been found that treatment of taste-receptor expressing cells with pertussis toxin (PTX), which blocks functional coupling between, GPCRs and Gi proteins, prevents MAPK activation and prevents a decrease in cAMP accumulation. These results indicate that the subject MAPK and cAMP assay systems provide an efficient means for identifying compounds that modulate, e.g., enhance, agonize or antagonize the activity of specific taste receptors i.e., T1R2/T1R3 (sweet receptor) or T1R1/T1R3 (umami receptors) or specific T2Rs (bitter receptors).

The subject MAPK assays are exemplified by the above-described antibody-based methods for detecting MAPK activation. As noted supra, however, the invention encompasses any suitable assay system for detecting activated MAPK. (71) Vaster et al., Biochem J. 350:717-22 (2000), incorporated by reference in its entirety herein, describes a phosphospecific cell-based ELISA for detecting p42/p44 MAPK, p38MAPK, protein kinase B and cAMP response-element binding protein. This assay, referred to as “PACE”, (phosphospecific antibody cell-based ELISA) detects activated MAPK without the use of radioactive labels, and can use adherent cells or cells in suspension.

Alternatively, the detection of MAPK activation can be effected by the use of proximity assays (AlphaScreen™) from Packard or by use of High Content Screen System (ERK MAPK Activation HitKit™) from Cellomics. These assays or other available MAPK assays, can be used as part of a high throughput screening platform for identifying bitter, sweet and umami receptor agonists and antagonists.

In the preferred embodiment, cAMP accumulation is measured by an immunofluorescence assay as described in the examples. However, as noted supra, the subject invention embraces the use of any suitable means for detecting cAMP levels. Such methods include the detection of cAMP using anti-cAMP antibodies in an ELISA-based format, or by second messenger reporter system assays. Promoters on genes drive the expression of the proteins that a particular gene encodes. Cyclic AMP drives gene expression by promoting the binding of a cAMP-responsive DNA binding protein or transcription factor (CREB) that then binds to the promoter at specific sites called cAMP response elements and drives the expression of the gene. As discussed supra, reporter systems can be constructed which have a promoter containing multiple cAMP response elements before a reporter gene, e.g., beta-galactosidose or luciferase. In this assay, a constitutively activated Gᵢ linked receptor causes a reduction in cAMP that results in inhibition of the gene expression and reduced expression of the reporter gene. The reporter protein can be detected using standard biochemical assays.

### Functional Coupling of Gᵢ Proteins to T1Rs and T2Rs

In another aspect, the present invention relates to the discovery that T1Rs and T2Rs functionally couple to G proteins other than promiscuous G proteins such as Gα₁₅ or gustducin. Particularly, the invention involves the discovery that T1Rs and T2Rs functionally couple to Gᵢ proteins and use G_{αi} to transmit signals to downstream effectors, e.g., adenylyl cyclase and MAP Kinase.

Gₛ stimulates the enzyme adenylyl cyclase. By contrast, Gᵢ (and G_{z} and G₀) inhibit this enzyme. Adenylyl cyclase catalyzes the conversion of ATP to cAMP. Thus, constitutively activated GPCRs that couple Gᵢ (or G_{z} and G₀) protein associated with a decrease in cellular levels of cAMP. See, generally, “Indirect Mechanisms of Synoptic Transmission,” Chapter 8, From Neuron to Brain (3rd Edition), Nichols, J. G. et al etds., Sinaver Associates, Inc. (1992). Thus, assays that detect cAMP can be utilized to determine if a compound is e.g., an inverse agonist to, the receptor (i.e., such a compound would decrease the levels of cAMP): As noted previously, a variety of approaches can be used to measure cAMP, e.g., anti-cAMP antibodies in an ELISA method, or the second messenger reporter system assays described supra.

As noted, a Gᵢ protein coupled receptor is known to inhibit adenylyl cyclase, resulting in a decrease in cAMP production. Another effective technique for measuring the decrease in production of cAMP as an indication of constitutive activation of a receptor that predominantly couples. Gᵢ upon activation can be accomplished by co-transfecting a signal enhancer, e.g., a non-endogenous, constitutively activated receptor that predominantly couples with Gₛ upon activation with the Gᵢ linked GPCR, i.e., a T1R or T2R. In contrast to Gᵢ coupled GPCRs, constitutive, activation of a Gₛ coupled receptor can be determined based upon an increase in production of cAMP. Thus, this construction approach is intended to advantageously exploit these “opposite” effects. For example, co-transfection of a non-endogenous, constitutively activated Gₛ coupled receptor (“signal enhancer”) with the Gᵢ coupled receptor (T1R or T2R) provides a baseline cAMP signal (i.e., although the Gᵢ coupled receptor will decrease cAMP levels, this “decrease” will be relative to the substantial increase in cAMP levels established by constitutively activated Gₛ coupled signal enhancer). By then co-transfecting the signal enhancer with a constitutively activated version of the target receptor, cAMP will decrease further (relative to the baseline) due to the increased functional activity of the Gᵢ target, i.e., T1R or T2R, which decreases cAMP.

Screening for potential T1R or T2R modulators using such a cAMP assay can then be accomplished with two provisos: first, relative to the Gᵢ coupled target receptor (T1R or T2R), “opposite” effects will result, i.e., an inverse agonist of the Gᵢ coupled target receptor will decrease this signal; second candidate modulators that are identified using this approach should be assessed independently to ensure that these compounds do not target the signal enhancing receptor (this can be accomplished prior to or after screening against co-transfected receptor).

Additionally, as described above, other assays can be designed which assess the effects of cAMP on other cellular events. Alteration of the intracellular concentration of cAMP is known to affect many cellular reactions. For example, an increase in cAMP intracellular concentrations stimulates the activity of protein Kinases. For a general review of cAMP and secondary messenger systems associated therewith, reference is made to “Molecular Cell Biology”, Darnell et al, Chapter 16 (1986) (156).

Particular signal substances that use cAMP as a second messenger include by way of example calcitonin, chorionic gonadotropin, corticotrophin, epinephrine, follicle-stimulating hormone, glucagon, leutenizing hormone, lipotropin, melanocyte-stimulating hormone, norepinephrine, parathyroid hormone (PTH), thyroid-stimulating hormone and vasopressin.

The subject assays which measure the effect of a putative modulator or TR/Gᵢ associated signaling pathways were not suggested by the state of the art. In vivo, receptors for bitter and sweet taste functionally couple to the taste-specific G-protein α-gustducin to initiate the transduction cascade leading to taste perception. In heterologous cells, however, previously there was no direct evidence of functional coupling to G-proteins other than Gα₁₅, a promiscuous G-protein widely used for receptor deorphaning. Unexpectedly, the present inventors have demonstrated that receptors for bitter, sweet and also umami taste couple effectively to Gᵢ-signaling pathways when expressed in human embryonic kidney cells. For example, cycloheximide, a bitter compound, specifically activates ERK1/2 mitogen-activated kinases in cells expressing the mouse bitter receptor mT2R5 and the rat bitter receptors rT2R9. Consistent with the foregoing, activation of ERK1/2 is totally abolished upon treatment with pertussis toxin indicating that these receptors couple to ERK1/2 activation through Gαᵢ. Also in agreement with these observations, cycloheximide inhibits the forskolin-induced cAMP accumulation in mT2R5-expressing cells by 70%. Similarly, as shown infra in the examples, natural and artificial sweeteners such as sucrose, D-tryptophan, saccharin and cyclamate (known activates of T1R2/T1R3 sweet receptors) activate ERK1/2 in cells expressing the human sweet receptor hT1R2/hT1R3. Also, as shown in detail infra, monosodium glutamate exclusively activates ERK1/2 in cells expressing the human umami receptor hT1R1/hT1R3 and the effect is greatly enhanced by the presence of inosine monophosphate. Again, consistent with Gi coupling, these responses are prevented by treatment with pertussis toxin.

Further, as shown in detail infra, sweeteners including cyclamate, aspartame, saccharin, and monellin significantly inhibit the forskolin-induced cAMP accumulation in hT1R2/hT1R3-expressing cells by 50-70%. Monosodium glutamate also decreases basal levels of cAMP in hT1R1/hT1R3-expressing cells by 50%.

While the results obtained are unexpected, some earlier information relating to taste-specific GPCRs is consistent with these results. Particularly it was known that taste-specific GPCRs use heterotrimeric G proteins to relay intracellular signals leading to cell depolarization and, subsequently, taste perception. Also, it was known that deletion of the gene encoding a taste-specific G protein subunit, α-gustducin (73) McLaughlin et al., Nature 357:563-569 (1992)), produces mice that are defective in detection of bitter and sweet substances (17). The visual G-protein α-transducin is also expressed in taste tissue (74, 75) (Ruiz-Avila et al., Nature 376:80-85 (1995); McLaughlin et al., Phys. Behav. 56(6):1157-64 (1994)) and its selective expression in α-gustducin deficient TRCs partially rescues the tasting phenotype to sweet and bitter substances (27) He et al, Chem. Senses, 27(8): 719-27 (2002). In biochemical assays, bitter tasting substances activate α-gustducin (127) Ming et al., Proc. Natl Acad Sci, USA 95(10): 8933-8 (1992) and α-transducin (128) Ruiz-Avila et al., Chem Senses, 20(4): 361-8 (2000). Similarly, cycloheximide induces α-gustducin activation in cell membranes derived from mT2R5-expressing cells Chandrashekar et al. Cell 100(6): 703-711 (2000) (80).

It has also been suggested that taste GPCRs use G-proteins other than α-gustducin to relay intracellular signals and that TRCs express a vast repertoire of different G protein subunits. Expression of Gαₛ, Gα₁₅, Gαᵢ₁₋₁, Gαᵢ₁₋₂, Gα₁₋₃ and Gα_{q} has been detected in taste tissues using RT-PCR (15, 25). Gαᵢ₁₋₂ can also be detected by in situ hybridization (25, 26) and immunostaining (25) in TRCs and a study by Hoon et al., (32) reported that Gᵢ proteins are expressed in almost all TRCs. As a result, Gαᵢ₁₋₂ positive cells are thought to be larger in number than Gα-gustducin-positive cells in rat circumvallate papillae (Kusakabe et al., Chem. Senses 25(5):525-31 (2000) (25)). Also, α-gustducin deficient mice retain residual responsiveness to bitter and sweet stimuli (Wong et al., Nature 381:796-800 (1996); He et al, Chem Senses 27(8): 719-27 (2002); Ruiz-Avila et al, PNC Natl Acad Sci, USA 98(15): 541-551 (2001) (17, 27, 28)) suggesting that another G protein may complement α-gustducin functions in TRCs.

Further, some earlier biochemical studies have suggested the possible existence of signaling pathways parallel to α-gustducin in TRCs. For example, the application of bitter-tasting substances (Kan et al., Am J. Physiol Cell Physiol. 280(2): C742-751 (2001) (76)) to taste tissue reduces the levels of 3′,5′-cyclic nucleotide monophosphate (cAMP) in taste tissue papillae. By contrast, the application of sweeteners to taste tissue membranes has been reported to increase levels of cAMP Naim et al, Comp. Biochem Physiol B 100(3): 455-§, (1991); Striem et al, Biochem J. 260(1): 121-6 (1989) (77, 78). However, prior to this invention there existed no direct evidence of functional coupling between taste GPCRs and G-proteins other than α-gustducin, α-transducin and Gα_{15/16}, a promiscuous G-protein widely used for receptor deorphaning (79) (Kostensis, Trends Pharmacol Sci 22(11) 560-564 (2001)). and none of these G-proteins were known to directly activate effectors capable of modulating the levels of cyclic nucleotides in TRCs.

Current models that do not take into account the experimental results herein suggested that the sweet taste receptor can also couple to Gαₛ and that α-gustducin activates, by unknown mechanisms, a taste specific cyclic nucleotide phosphodiesterase (PDE) (9, 10) (Gilbertson et al., Curr. Opin. Neurobiol. 10(4): 519-27 (2000); Margolskee, R. F., J. Biol Chem 277(1):1-4 (2002)). However, these hypothetical signaling pathways have not yet been definitely linked to taste receptor activation in TRCs or in fact any other cell types.

By contrast, the present inventors have studied coupling of receptors for bitter, sweet and umami taste to classical GPCR-linked signaling pathways in HEK293 cells, and the results obtained surprisingly demonstrate that these taste receptors can effectively couple to Gαᵢ-dependent activation of mitogen activated protein (MAP) kinases ERK1 and ERK2 (ERK1/2) and Gαᵢ-dependent inhibition of cAMP accumulation. Also, these results further surprisingly indicate that the sweet receptor does not couple to Gₛ stimulation and accumulation of cAMP. Functional coupling to Gαᵢ may explain, in part, the observations that bitter-tasting substances and MSG decrease the level of cyclic nucleotides in TRCs. Moreover, these results suggest that Gαᵢ can functionally complement α-gustducin functions in TRCs.

### Applications of the Subject Assays

The present invention provides cell-based assay methods that rely on the discovery that T1Rs and T2Rs functionally couple to Gᵢ proteins e.g., Gαᵢ and transmit signals to downstream effectors, e.g., cAMP, MAP Kinase, and adenylyl cyclase that enable the identification of modulators, e.g., agonists, antagonists, inverse agonists enhancers of a T1R or T2R polypeptide. The T2R modulators of the invention are useful for altering taste perception, for example to induce, suppress or enhance bitter taste perception in a subject. The T1R2/T1R3 modulators are useful for modulating sweet taste, e.g., by enhancing the taste of another sweet tasting compound such as saccharin. The T1R1/T1R3 modulators identified according to the invention are useful for modulating umami taste, e.g., by enhancing the taste of a umami compound such as monosodium glutamate.

### Compositions

In accordance with the methods of the present invention, a composition that is administered to alter taste perception in a subject will comprise an effective amount of a T1R or T2R modulator (agonist, antagonist, or enhancer). A T1R or T2R activator or modulator can comprise any substance e.g., small molecule, peptide, protein, carbohydrate, oligosaccharide, glycoprotein, amino acid derivative, and the like. In general, compounds will be identified by screening libraries of potential taste modulatory compounds, which may be comprised of synthetic or naturally occurring compounds. The library may be random or may comprise compounds having related structures or are structures or substitutions. After lead candidates are identified, compound libraries having similar structure will be produced and screened for T1R or T2R modulatory activity according to the invention. T1R or T2R modulators identified as disclosed herein can be used to prepare compositions suitable for oral use, including but not limited to food, beverages, oral washes, dentifrices, cosmetics, and pharmaceuticals. T1R or T2R modulators can also be used as additives to alter the sweet, umami or bitter taste of a compound that is of palatable but undesirable for oral use, for example compounds comprised in household cleansers, poisons, etc. Such modulators will alter bitter, sweet or umami tasting compounds contained therein.

For example, representative foods having an undesirable or bitter taste include, but are not limited to, citrus fruits such as grapefruit, orange, and lemon; vegetables such as tomato, pimento, celery, melon, carrot, potato, and asparagus; seasoning or flavoring materials such as flavor, sauces, soy sauce, and red pepper; foods originating from soybean; emulsion foods such as cream, dressing, mayonnaise, and margarine; processed marine products such as fish meat, ground fish meat, and fish eggs; nuts such as peanuts; fermented foods such as fermented soybean; meats and processed meats; pickles; noodles; soups including powdery soups; dairy products such as cheese; breads and cakes; confectioneries such as candies, chewing gum, and chocolate; and specifically prepared foods for health.

Representative cosmetics eliciting bitter taste (e.g., skin lotions, creams, face packs, lip sticks, foundations, shaving preparations, after-shave lotions, cleansing foams, and cleansing gels) include but are not limited to those compositions that include surfactants such as sodium alkyl sulfate and sodium monoalkyl phosphate; fragrances such as menthol, linalool, phenylethyl alcohol, ethyl propionate, geraniol, linalyl acetate and benzyl acetate; antimicrobials such as methyl paraben, propyl paraben and butyl paraben; humectants such as lactic acid and sodium lactate; alcohol-denaturating agents such as sucrose octaacetate and brucine; and astringents such as aluminum lactate.

Representative pharmaceuticals having a bitter taste include acetaminophen, terfenadine, guaifenesin, trimethoprim, prednisolone, ibuprofen, prednisolone sodium phosphate, methacholine, pseudoephedrine hydrochloride, phenothiazine, chlorpromazine, diphenylhydantoin, caffeine, morphine, demerol, codeine, lomotil, lidocaine, salicylic acid, sulfonamides, chloroquine, a vitamin preparation, minerals and penicillins, neostigmine, epinephrine, albuterol, diphenhydramine, chlorpheniramine maleate, chlordiazepoxide, amitriptyline, barbiturates, diphenylhydantoin, caffeine, morphine, demerol, codeine, lomotil, lidocaine, salicylic acid, sulfonamides, chloroquine, a vitamin preparation, minerals and penicillins.

Representative sweeteners which may be modulated by compounds according to the invention include xylitol, sorbitol, saccharin, sucrose, glucose, fructose, cyclamate, aspartame, monellin, and the like, and derivatives thereof.

Representative umami compounds, the taste which may be modulated according to the invention include L-glutamate, L-asparate, monosodium glutamate, derivatives thereof, compounds containing and the like.

These taste modulators can also be administered as part of prepared food, beverage, oral wash, dentifrice, cosmetic, or drug. To prepare a composition suitable for administration to a subject, a T1R or T2R modulator can be admixed with a compound, the taste of which is to be modulated in amount comprising about 0.001% to about 10% by weight, preferably from about 0.01% to about 8% by weight, more preferably from about 0.1% to about 5% by weight, and most preferably from about 0.5% to about 2% by weight.

Suitable formulations include solutions, extracts, elixirs, spirits, syrups, suspensions, powders, granules, capsules, pellets, tablets, and aerosols. Optionally, a formulation can include a pharmaceutically acceptable carrier, a suspending agent, a solubilizer, a thickening agent, a stabilizer, a preservative, a flavor, a colorant, a sweetener, a perfume, or a combination thereof. T1R or T2R modulators and compositions can be presented in unit-dose or multi-dose sealed containers, such as ampules and vials.

### Administration

T1R or T2R modulators can be administered directly to a subject for modulation of taste perception. Preferably, a modulator of the invention is administered orally or nasally.

In accordance with the methods of the present invention, an effective amount of a T1R or T2R modulator is administered to a subject. The term “effective amount” refers to an amount of a composition sufficient to modulate T1R or T2R activation and/or to modulate taste perception, e.g., bitter, sweet or umami taste perception.

An effective amount can be varied so as to administer an amount of an T1R or T2R modulator that is effective to achieve the desired taste perception. The selected dosage level will depend upon a variety of factors including the activity of the T1R or T2R modulator, formulation, combination with other compositions (e.g., food, drugs, etc.), the intended use (e.g., as a food additive, dentifrice, etc.), and the physical condition and prior medical history of the subject being treated.

An effective amount or dose can be readily determined using in vivo assays of taste perception as are known in the art. Representative methods for assaying taste perception are described infra.

### EXAMPLES

The invention is further illustrated by the following non-limiting examples wherein the following materials and methods are used.

### Materials and Methods

Sweeteners, agonists and toxins. Sucrose, aspartame, cyclamate, monellin, monosodium glutamate, inosine monophosphate, isoproterenol, epidermal growth factor, denatonium benzoate, quinine sulfate, cycloheximide, rolipram and forskolin were from Sigma (St-Louis, Mo.). Pertussis toxin (PTX) was from List Biological Laboratories (Campbell, Calif.).

Establishment of stable cell lines. An inducible expression system was used for the umami taste receptor line (hT1R1/hT1R3). Vectors, were prepared using the GeneSwitch inducible system (Invitrogen, Carlsbad, Calif.). hT1R1 and hT1R3 vectors were prepared by cloning receptor cDNA into pGene/V5-His A at EcoRI/Not I sites. A modified pSwitch vector was also prepared by replacing the hygromycin β resistance gene with the puromycin resistance gene. The cDNAs for hT1R1, hT1R3, and puromycin resistance were co-transfected into HEK293 cells stably expressing Gα₁₅ (Aurora Biosciences, San Diego, (80) Chandraskekar et al, Cell 100(6): 703-11 (2000). hT1R1/hT1R3 stable cell lines were selected and maintained in high-glucose DMEM media containing 100 μg/mL zeocin, 0.5 μg/mL puromycin, 2 mM GIutaMAX 1, 10% dialyzed fetal bovine serum, 3 μg/mL blasticidin and penicillin/streptomyocin. To improve cell adhesion, cell flasks were pre-coated with Matrigel (Becton-Dickinson, Bedford, Mass.) at a dilution of 1:400. Expression of hT1R1 and hT1R3 was induced by treatment of cells with 6×10⁻¹¹M mifepristone for 48 hours prior to experiments. Clones were tested and selected for mifepristone-induced responsiveness to MSG/IMP using calcium-imaging experiments (data not shown). The clone used in this study did not show any functional expression of hT1R1/R3 without induction (data not shown).

Establishment of the sweet (hT1R2/R3) receptor line stable cell line has already been described Li et al., Proc. Natl Acad. Sci, USA 99(7): 4692-6 (2002) (14). Cells were maintained in low-glucose DMEM media containing 10% heat-inactivated dialyzed FIBS, penicillin/streptomyocin, 3 μg/mL blasticidin, 100 ug/ml zeocin, and 0.5 ug/ml puromycin in Matrigel-coated flasks.

HEK293 cells were transfected with 5 μg of linearized Rho-mT2R5 plasmid (80) Chandraskekar et al (2000) in pEAK10 (Edge biosystems) using the Transit transfection reagent (Panvera). Cells were selected in the presence of 0.5 μg/ml puromycin, clones were isolated, expanded and analyzed by fluorescence-activated cell sorting for the presence of Rho tag immunoreactivity at the cell surface using a monoclonal antibody; raised against the first 40 amino acids of rhodopsin (80, 81) (Chandrashekar et al (2000); Adamus et al., Vision Res. 31(1): 17-31 (1991)).

### Example 1

### MAP Kinase Assays

Transient transfection of HEK293 cells for ERK112 assay. Subclonfluent HEK293 cells in 10 cm dishes were transfected with 4 μg of Rho-rT2R9 plasmid (Chandrashekar et al (2000); Bufe et al., J. Receptor Signal Transduct. Res. 20(2-3): 153-166 (2000)) pEAK10 (Edge Biosystems, Gaithersburg, Md. (80, 82)) and 2 μg pUC-18 as a carrier DNA using the Transit transfection reagent (Panvera). 24 hours later, cells were harvested using Hank's balanced salt solution without calcium or magnesium and containing 1 mM EDTA (HBSS/EDTA), and plated into 6 well plates. ERK1/2 assay was performed 48 hours post-transfection.

Determination of ERK112 phosphorylation Cells were seeded into matrigel-coated 6-well plates at a density of 0.4-0.8 million cells per well 48 hours prior to experiment. When necessary, receptor induction was initiated on the same day with 6×10−1 IM mifepristone. 16 hours prior to experiment, cells were starved using serum-free growth media containing 1% fatty acid-free bovine serum albumin (Sigma, St-Louis, Mo.). Cells were then stimulated with 2× agonist solutions in HBSS or Dulbelcco's phosphate buffered saline (D-PBS) (Invitrogen, Carlsbad, Calif.) for 5 minutes at 37° C. Following stimulation, cells were placed on ice and washed once with ice-cold buffer. Lysis buffer containing 150 mM NaCl, 50 mM Tris HEl pH 8, 0.25% sodium deoxycholate, 1% igepal (NP-40), 2 mM sodium orthovanadate, 1 mM sodium fluoride, and protease inhibitors were then added and cells were scraped off the plates. Lysates were frozen immediately in liquid nitrogen and kept at −80° C. until further analysis.

Lysate protein concentration was determines using the Bradford method (Amresco, Solon, Ohio). Cell lysate proteins (22 FLg/lane) were resolved by SDS-PAGE using 4-20% Tris-glycine gels (Invitrogen, Carlsbad, Calif.). Following electrophoresis, proteins were transferred to nitrocellulose membranes that were subsequently blocked with 5% fat-free milk in Tris-buffer saline containing 0.2% tween-20 (TBST). Membranes were immunoblotted with phospho-p44/42 MAPK monoclonal antibody (Cell Signaling Technology, Beverly, Mass.) diluted 1:1000 in 5% milk/TBST overnight at 4° C. Secondary antibody was HRP-linked anti-mouse IgG diluted 1:2000 in 5% milk/TBST. Immunoreactive proteins were revealed using SuperSignal ECL solution (Pierce Chemical, Rockford, Ill.). Results were quantified using Kodak Image Station 440CF. In all experiments, we also assessed total amount of p44/42 MAPK loaded in each lane.

Membranes were stripped of phospho-specific antibodies using 0.2 M glycine pH 2.5 and re-blotted with p44/42 polyclonal antibodies (Cell Signaling Technology, Beverly, Mass.) diluted 1:1000 in 5% milk/TBST overnight at 4° C. Secondary antibody was HRP-linked anti-rabbit IgG diluted 1:2000 in 5% milk/TBST.

### Example 2

### cAMP Experiments

cAMP content of cells was determined by a commercially-available chemiluminescent immunoassay kit (Applied Biosystems, Foster City, Calif.). Assay plates (96-well) were precoated with matrigel at a dilution of 1:400, and cells were seeded at a density of 60,000 cells/well (mT2R5), 75,000 cells/well (hT1R2/hT1R3) and 50,000 cells/well (hT1R1/R3) 48 hours prior to experiment. Induction of hT1R1/R3 expression was also initiated 48 hours prior to experiment. Cell media was aspirated and 90 μl of pre-warmed HBSS or D-PBS was added to each well. Cells were incubated for 45 minutes at 37° C., buffer was aspirated and 90 μl of pre-warmed agonist solutions in HBSS or D-PBS containing 50 μM rolipram and 0.7 to 5 μM forskolin was added to each of the corresponding wells. Plates were incubated for 15 minutes at 37° C. Agonists were aspirated and stimulation was terminated with addition of 60 μl of lysis buffer into each well. cAMP levels were then determined as described by the kit instructions. An independent cAMP standard curve was performed on each 96-well plates used. Chemiluminescent signals were detected using a TopCount NXT (PerkinElmer, Wellesley, Mass.) set at a read-time of 2 seconds/well.

### Example 3

### Taste Study

A flavor acceptance study is conducted using a test composition comprising a T1R or T2R modulator identified according to the foregoing examples. A control composition lacking the T1R or T2R modulator, but which is otherwise substantially similar or identical to the test composition, is also used. The study employs a two-way crossover design, with all subjects evaluating both compositions, which are administered in one or more same amounts or doses. The test and control compositions are evaluated on a single study day. The sequence for administering the test and control compositions is randomized among subjects. All enrolled subjects complete all aspects of the study protocol. Subjects respond to each of the test and control compositions using ordinal taste scores (e.g., in the case of a putative T2R modulator 1=very bitter, 2=bitter, 3=indifferent, 4=not that bitter, 5=not bitter at all). Adverse events are recorded. Effectiveness of a T1R or T2R modulator is determined by measuring a significant difference in palatability of the test composition when compared to the control composition.

### Results

The results of the MAP Kinase assays described supra demonstrate that the sweet and umami receptors activate ERK1/2 in a pertussis toxin sensitive fashion. The inventors used mT2R5, a mouse bitter receptor that recognizes cycloheximide (80) Chandrashekar et al. (2000), and the hT1R2/hT1R3 (hT1R2/R3) and hT1R1/hT1R3 (hT1R1/R3) combinations, the recently identified human receptors for sweet (14, 15) and MSG (umami) taste (14, 15) (Li et al (2002); Nelson et al (2002)) respectively. A clone stably expressing mT2R5 shows robust induction of ERK1/2 phosphorylation upon exposure to cycloheximide (FIG. 1A). Activation of ERK1/2 by cycloheximide in mT2R5-expressing cells peaks at 3-5 minutes post-stimulation (FIG. 1B). Other bitter substances including quinine and denatonium benzoate, sweeteners such as saccharin or sucrose and MSG do not induce. ERK1/2 activation in mT2R5-expressing cells (FIG. 1A). Similarly, stimulation of rT2R9, the rat receptor orthologue of mT2R5 (85) Bufe et al, Nat. Genet. 32(3): 397-401, with cycloheximide leads to ERK1/2 activation in transiently transfected HEK293 cells (FIG. 1C). Sweeteners such as sucrose, saccharin, cyclamate and the sweet tasting amino acid D-tryptophan activate ERK1/2 in hT1R2/R3-expressing cells (FIG. 2A). Here again, the effect is specific for sweeteners as bitter substances and MSG fail to activate ERK1/2 in hT1R2/R3-expressing cells (FIG. 2B). MSG induces ERK1/2 activation in hT1R1/R3 expressing cells (FIG. 2B). Sweeteners and bitter substances have no significant effect on the level of activated ERK1/2 in these cells (FIG. 2B). The effects of cycloheximide on mT2R5, of saccharin, cyclamate, D-tryptophan and sucrose on hT1R2/R3 and of MSG on hT1R1/R3 are receptor dependent since naive cells do not respond significantly to any of these modalities (FIG. 1E and FIG. 2C and results not shown).

Cycloheximide activates ERK1/2 in a dose-dependent fashion in mT2R5-expressing cells with an EC₅₀ of 1.1+/−0.4 μM (mean+/−SD of three independent determinations) (FIG. 1D). Saccharin and sucrose also induce ERK1/2-activation in a dose-dependent fashion in hT1R2/R3-expressing cells (FIGS. 3A and 3B). As expected from taste thresholds (14) (Li et al (2002)), saccharin is much more potent with an EC₅₀ of 277+/−47 RM compare to an EC₅₀ of 73+/−37 mM for sucrose (mean+/−SD of three independent determinations) (FIGS. 3A and 3B). One of the hallmarks of umami taste is its spectacular enhancement by inosine monophosphate (IMP) (86) Yamaguchi et al, Physiol. Behav. 49(5): 833-841 (1991). Accordingly, in the ERK1/2 assay, we observe a leftward shift of MSG EC₅₀ of about 30 folds in presence of 10 mM IMP (FIG. 3C) (EC₅₀ MSG: 6.7+/−3.4 mM, EC₅₀ MSG in the presence of 10 MM IMP: 0.4+/−0.3 mM; mean+/−SD of three independent determinations). PTX has been widely used as a powerful tool to discriminate among the different pathways used by GPCRs to activate ERK1/2 (87) Liebmann et al., Cell Signal 13(11): 833-41 (2001). Treatment of HEK293 cells with PTX prevents stimulation of ERK1/2 by cycloheximide (FIG. 2A), by sucrose, saccharin, D-tryptophan and cyclamate (FIG. 2A) and by MSG (FIG. 2B) without affecting the response of epidermal growth factor (EGF), a known tyrosine kinase receptor agonist. Collectively, these results indicate that taste receptors functionally couple to G; proteins to induce ERK1/2 activation in HEK293 cells.

Activation of bitter, sweet and umami receptors inhibit cAMP accumulation in HEK293 cells. Results described in FIGS. 2 and 3 suggest that taste receptors should also functionally couple to an inhibition of adenylyl cyclase and a reduction of cAMP levels in HEK293 cells. FIG. 4A shows that cycloheximide leads to a 70% reduction of forskolin-induced cAMP accumulation in mT2R5-expressing cells. In agreement with the involvement of G; proteins, PTX treatment fully abolishes the inhibition (FIG. 5A). The effect of cycloheximide on cAMP accumulation is mT2R5-dependent since cAMP levels remain unchanged if the same experimental conditions are applied on naive HEK293 cells (FIG. 4A). Cycloheximide inhibits cAMP accumulation in a dose-dependent fashion in mT2R5-expressing cells with an EC₅₀ of 1.2+/−0.7 μM (FIG. 5A) (mean+/−SD of three independent determinations) a value similar to the EC₅₀ calculated for ERK1/2 activation (FIG. 5D). The sweet taste hT1R2/R3 receptor also functionally couples to a robust inhibition of cAMP accumulation in HEK293 cells. Sweeteners such as aspartame, cyclamate, saccharin and monellin decrease forskolin-induced cAMP accumulation levels by 55%, 40%, 55% and 64% respectively and in a PTX-sensitive fashion (FIG. 5A). Fructose and sucrose do not inhibit cAMP accumulation in hT1R2/R3-expressing cells, on the contrary; fructose apparently increase cAMP levels (FIG. 5A). The lack of apparent effect of fructose and sucrose in the inhibition assay can be explained by the fact that these two sweeteners consistently increase cAMP levels in HEK293 cells not expressing the sweet receptor (FIG. 5B). Cyclamate (FIG. 5C), aspartame (FIG. 5D) and saccharin (FIG. 5E) inhibit cAMP accumulation in a dose-dependent fashion with EC₅₀s of 1.2+/−0.7 mM, 350+/−60 μM and 61+/−33 μM respectively (FIG. 5C) (mean+/−SD of three independent determinations). Our hT1R1/hT1R3 umami taste receptor line exhibits a very high basal cAMP level relative to our mT2R5 and hT1R2/hT1R3 lines (mT2R5 line: 2.8+1-1.9 pmol/well, T2R2/R3 line: 4.5+/−1.9 pmol/well, hT1R1/hT1R3 line: 180+/−30 pmol/well). Under experimental conditions similar to the one used for the mT2R5 and hT1R2/hT1R3 lines (in the presence of forskolin), cAMP levels more than often reached non-linear range values with the hT1R1/hT1R3 line (results not shown). However, in the absence of forskolin, MSG decreases basal levels of cAMP by 50% in this cell line (FIG. 6). On the other hand, cAMP levels remain unchanged even in the presence of MSG when receptor expression is not induced (FIG. 6).

Sweet and bitter receptors do not couple to Gₛ-stimulation in HEK293 cells. Current models suggest that the sweet receptor may couple to GS to increase cAMP levels in TRCs (9, 10) (Gilbertson et al (2000); Margolskee (2002)). Clearly, our results with ERK1/2 activation and inhibition of cAMP accumulation point to a direct coupling to Gᵢ proteins (FIGS. 2, 3 and 5). However, it is still possible that this receptor could have dual properties, coupling to both Gᵢ and Gₛ. Therefore, we sought to determine if we could detect an agonist-induce increase in cAMP, levels in the hT1R2/R3 sweet taste receptor line. Under these experimental conditions (i.e. in the absence of forskolin), cAMP levels remain unchanged after stimulation with aspartame, cyclamate, saccharin and monellin (FIG. 7A). On the other hand, a β-adrenergic receptor (β2AR) agonist, isoproterenol, induces a 100% increase of cAMP accumulation in hT1R2/hT1R3-expressing cells indicating that a functional receptor/Gₛ interection can be detected under these experimental conditions. The sweeteners do not induce an increase of cAMP levels even after inhibiting functional coupling to Gᵢ With PTX (FIG. 7B). On the other hand, the isoproterenol response increases significantly (by more than 17 fold) under these conditions, confirming that the β2AR couples to both Gᵢ and Gₛ proteins in HEK293 cells (88) (Paaka et al, Nature 390:88-91 (1997). Our experiments with mT2R5 suggest that bitter receptors do not functionally couple to Gₛ either. Cycloheximide does not increase levels of cAMP in HEK293 cells, even after inhibiting coupling to Gᵢ proteins with PTX (FIG. 7C). Interestingly, inhibiting functional coupling to Gᵢ with PTX in the umami taste hT1R1/hT1R3 line uncovers a modest increase of 25% in cAMP levels (FIG. 6). Further experiments are necessary to determine if hT1R1/hT1R3 can indeed couple to Gₛ signaling pathways in a significant fashion.

### CONCLUSIONS

In this application, the present inventors have investigated the functional coupling of taste receptors to ERK1/2 activation and to the modulation of intracellular cAMP levels, two classical signaling events activated by dozens of GPCRs (89, 90, 91) (Morris et al., Physiol. Rev. 79(4): 1373-1430 (1999); Chin et al., Ann. NY Acad. Sci. 968: 49-64 (2002); Liebmann et al, J. Biol. Chem. 271(49): 31098-31105 (1996)). cAMP is a universal second messenger used by a plethora of cell surface receptors to relay signals from the extracellular milieu to the intracellular signaling machinery such as protein kinases, transcription factors and ion channels (89, 90, 92) (Morris and Malbon (1999); Chin et al (2002); Robinson-White and Stratakis, Ann NY Acad. Sci. 968: 256-270 (2002)). GPCRs activation of Gαs and Gαᵢ respectively increase and decrease intracellular cAMP levels (Hanoune and Defer, Annu Rev. Pharmacol. Toxical 42: 145-174 (2001) (39)) (Hansom and Defr (2001)). The GTP-bound form of Gαₛ directly interacts and activates the 9 types of membrane-bound adenylyl cyclase (AC) known (93). Conversely, the GTP-bound form of Gαᵢ can directly interact and inhibit up to 6 different types of AC (39). ERK1/2 is activated by G_{q}, Gₛ and Gi-coupled GPCRs (Liebmann et al (1996); Pierce et al., Oncogene 20(13): 1532-1539 (2001); Gutkind, J. S., J. Biol Chem 273(4): 1839-42 (1998) (91, 94, 95)) and, depending on the cellular context, several signaling pathways can be triggered to activate ERK1/2. Specifically, it is thought that Gᵢ-coupled GPCRs activate ERK1/2 mainly via the free (activated) Gβγ subunits (Crespo et al. Nature 369: 418-20 (1994); Faure et al., J. Biol Chem. 269(11): 7852-7854 (1999) (96, 97)) that recruit and activate soluble tyrosine kinases of the Src (Gutkind, 1998 (95)) and Bruton families (Wan et al., J. Biol Chem. 272(27): 17209-15 (1997) (98)) or somehow transactivate receptor tyrosine kinases (RTKs) at the cell surface to initiate the cascade Liebmann et al. (2001); Wu et al. Bioch. Biophys Acta. 1582:100-106 (2002) (87, 99)).

We have shown that a rodent bitter receptor, mT2R5, the human sweet taste receptor, hT1R2/hT1R3, and the human umami taste receptor, hT1R1/R3, couples to the activation of ERK1/2 and the inhibition of cAMP accumulation in HEK293 cells. The bitter substance cycloheximide, the sweeteners saccharin sucrose, cyclamate, D-tryptophan and the flavory amino acid MSG activate ERK1/2 exclusively in cells expressing their respective receptors. The effects of cycloheximide on mT2R5, saccharin and sucrose on hT1R2/R3 and MSG on hT1R1/R3 reach saturation at higher concentrations and their potency at activating ERK1/2 is similar to the ones reported for the G₁₅-induced calcium mobilization in HEK293 (80, 14) (Chandrashekar et al (2000); Li et al., (2002)). Similarly, cycloheximide, artificial sweeteners, a sweet protein as well as MSG decrease cAMP levels exclusively in cells expressing their respective taste receptors. Here again, the effects are receptor dependent and the potency of these compounds at inhibiting cAMP accumulation is in agreement with taste thresholds and EC₅₀'s reported for the G_{α15}-induced calcium mobilization in HEK293 (Chandrashekar (2000); Li et al. (2002); Temussi et al. FEBS Lett. 526(1-3): 1-4 (2002) (80, 14, 100)). Collectively, these results indicate that bitter compounds, sweeteners and MSG specifically activate their taste receptors to induce ERK1/2 activation and the reduction of cAMP accumulation in heterologous cells.

α-subunits of the Gᵢ family including Gαᵢ₁₋₁, Gαᵢ₁₋₂, Gαᵢ₁₋₃, Gαᵢ₀₋₁, Gαᵢ₀₋₂, α-transducin and α-gustducin contain a conserved carboxyl-terminal cystein residue that is a site for modification by PTX, a 5′-diphosphate-ribosyltransferase isolated from Bortadella pertussis (101) (Fields et al. Biochem J. 321(P1-3): 561-71 (1997)). PTX specifically and irreversibly modifies these G-protein subunits in vivo with attachment of an ADP-ribose moiety and, as a result, this covalent modification physically uncouples the G-protein from activation by GPCRs (101) (Fields et al. (1997)). In our-assays, incubation of cells with PIX abolishes the activation of ERK1/2 by the bitter, sweet and umami taste receptors indicating that one or more members of the Gᵢ family functionally link the taste receptors to this signaling pathway in HEK293 cells. It is very likely that α-subunits of Gαᵢ₁₋₃ subfamily are involved since expression of Gαᵢ₁₋₂ is restricted to the brain (Offermanns, S. Naunyn Schmiedz Berg, Arch Pharmacol. 360(1): 5-13 (1999) (102)) and that α-transducin and α-gustducin expression is mostly restricted to the eye and the tongue (McLaughlin et al. (1994); Offermanns (1999) (75, 102)). Similarly, PTX prevents activation of ERK1/2 by other Gᵢ-coupled GPCRs expressed in HEK293 cells or different cell lines (Della Rocca et al (1997); Della Rocca et al (1999); Soeder et al., J. Biol Chem. 274(17): 12017-12026 (1999); Alderton et al, J. Biol Chem. 276(16): 13152-13460 (2001)—Alderton et al., Br. J. Pharmacol. 1341(1): 6-4 (2001) (83, 84, 103-105)). Every taste GPCR that we studied also couples to the inhibition of forskolin-induced cAMP accumulation in HEK293 cells and PTX-treatment totally abolishes the inhibition. This result clearly indicates that taste receptors directly couple to one or more member of the Gαᵢ₁₋₃ subfamily in these cells. In this signaling pathway, activated Gαᵢ proteins directly interact and inhibit the membrane bound adenylyl cyclase. There is indeed no evidence yet for direct regulation of cAMP-phosphodiesterases (PDEs) by the Gαᵢ₁₋₃ subfamily or, in fact, by any member of the Gi family (Hanoune and Defer (2001) (93)).

It has been postulated that cyclic nucleotides such as cAMP and cGMP are involved in taste transduction (10, Margolskee (2002)). Denatonium benzoate and strychnine, two extremely bitter substances, were shown to decrease the level of cAMP and cGMP in mouse taste bud homogenates (Yan et al. (2001) (76)). In 1995, Margolskee and colleagues reported the purification of a transducin-activated PDE activity from TRCs (Ruiz-Avila et al (2001) (28)). These results have inspired a model in which bitter taste receptors couple to α-gustducin/α-transducin that in turn couples to the activation of a PDE in TRCs (10). Gαᵢ subunits are highly expressed in TRCs (McLaughlin et al. (1994); Katsukobe et al. (2000); Asano-Miyoshi (2000) (75, 25, 26)). We propose, as depicted schematically in FIG. 8, that in addition to the hypothetical α-gustducin/α-transducin-PDE pathway, that bitter receptors may decrease intracellular levels of cAMP in TRCs through the direct inhibition of ACs by activated G_{αi5} (FIG. 8). It is not yet clear what could be the role of cAMP in TRCs functions. A decrease of cAMP in TRCs has been proposed to activate a cyclic nucleotide monophosphate (cNMP)-suppressible channel, leading to depolarization (Kolesnikov and Margolskee, Nature 376:80-88 (1995) (106)). (10) A recent study (Zhang et al., (2003) (18)) showing the essential requirement of the PLCβ2 pathway for the detection of sweeteners, bitter compounds and amino acids in rodents suggest that the cAMP pathway plays only a minor role in taste perception, if any. Still, modulation of cAMP levels in TRCs could have other effects than perception per se (FIG. 8). A recent report suggests that adrenergic transmission within the taste bud could play a paracrine role in taste physiology (29, Harness et al. (2002)). In this scenario, cAMP could have more of a modulator role, controlling intensity and/or the duration of taste sensation. In addition, the cAMP response element-binding protein (CREB) and phosphorylated-CREB have been recently localized in TRCs (55), suggesting that gene expression regulation can be potentially controlled, at least in part, by the level of cAMP in TRCs.

Over the past decade, three independent lines of observations had pointed to a potential role of cAMP in modulating sweet-taste signaling and sensation. First, early experiments showed that cAMP caused membrane depolarization of electrode-clamped mouse receptor cells (Tonosaki et al., Nature 331:304-6 (1988) (107)) and of patch-clamped frog receptor cells (Avanet et al, Nature 331:351-9 (1988)). Further investigation suggested that this depolarization could be mediated by a cAMP-dependent protein kinase inactivating an outward potassium current (Avanet et al (1988) (108)). Second, sweeteners and membrane permeant analogues of cAMP were shown to activate the same subset of hamster TRCs in vitro (Cummings and Kinnamon, J. Neurophysiol. 70(6): 2326-2336 (1993) (109)). In addition, just like cAMP (Avanet et al (1988) (108)), saccharin was shown to depolarize hamster and gerbil TRCs by reducing outward potassium currents (Cummings and Kinnamon, J. Neurophysiol. 75(3):1256-63 (1996); Uchida and Sato, Chem. Senses. 22(3): 163-164 (1997) (110, 111)). Lastly, sweeteners such as saccharin and sucrose were shown to increase cAMP levels in rat taste epithelium (Striem et al., 1989 (78)), in mouse fungiform taste buds (Nakashima and Ninomiya, Cell Physiol. Biochem. 9(2):90-98 (1999) (112)) and in pig circumvallate papillae (77, Naim et al., 1991). Together, these observations have led to the suggestion that the sweet receptor couples to Gₛ in TRCs (9, 10). In our hands, however, the sweet receptor clearly couples to a reduction of intracellular cAMP levels and activation of ERK1/2 through the direct functional coupling with Gᵢ. Moreover, we have consistently failed in detecting a sweetener-induced accumulation of cAMP, even after inhibiting functional coupling of hT1R2/R3 to Gᵢ proteins. It is noteworthy that we can detect a fructose or sucrose-induced cAMP accumulation in naive HEK293 cells. As mentioned above, we strongly suspect that this is a direct result of the osmotic shock triggered by the high concentrations of sucrose and fructose used in our experiments. Similarly, in an independent study, sucrose was shown to induce cAMP accumulation in tongue muscle membranes (Striem et al., (1989) (78)), a non-taste tissue. It is therefore possible that the sweeteners-induced increase in cAMP levels observed in rat taste epithelium (Striem et al (1989) (78)), in mouse fungiform taste buds (78) and in pig circumvallate papillae (Naim et al. (1991) (77)) occurs through a receptor-independent mechanism. In any case, our results do not support the hypothesis of a direct functional coupling of the sweet receptor to Gₛ (Gilbertson et al, 2000; Marlgokskee (2002) (9, 10)). The effect of MSG on the level of cyclic nucleotides in TRCs is much less understood. One report suggests that MSG induces a decrease in cAMP levels in circumvallate and foliate taste buds (Chaudhuri and Roper, Ann NY Acad. Sci. 855:398-406 (1998) (113)) while another report claims an increase in cAMP levels in fungiform papillae (Ninomiya et al., J. Nutr. 130 (3S Suppl):9500-9530 (2001) (114)). Our data clearly demonstrates that the umami receptor functionally couples to a reduction of intracellular cAMP levels and to the Gᵢ-induced activation of ERK1/2 in HEK293 cells. It is not known yet if the MSG (umami) receptor couples to α-gustducin in vivo. Our results point to Gαᵢ as a strong candidate for its cognate G protein in TRCs (FIG. 8).

These results suggest that gustducin is not the only Gα-subunit used for taste transduction. The level of co-expression in TRCs between T1R1 and T1R2 and α-gustducin is estimated at around −15% in rodents (Hoon et al., Cell 96(4): 541-551 (1999) (115)). Similarly, another study reported that only about 10% of T1R3 positive cells were also α-gustducin positive in mouse TRCs (Montmayeur et al., Nat. Neurosci. 4(5):492-498 (2001) (116)). Thus, in conclusion, most cells expressing the sweet and umami receptor subunits do not express α-gustducin. In consequence, one could expect that sweet and umami taste perception is mediated, in part, by a different G-protein. Perhaps the most compelling other evidence suggesting the involvement of other G-proteins is the residual responsiveness of α-gustducin deficient mice to bitter and sweet stimuli (Wang et al., (1996); He et al., (2002); Ruiz-Avila et al. (2001) (17, 27, 28)). A recent study shows that expression of a dominant negative form of α-gustducin, from the gustducin promoter in these deficient mice, further decreases the residual responsiveness to sweet and bitter stimuli, substantiating the notion on the involvement of another G protein (28, Ruiz-Avila et al., 2001). Independent studies report that umami (117) (Caicedo and Roper, J. Physiol. 544(pt 2): 501-509 (2002), sweet (117, 118) (Caicedo and Roper (2002); Bernhardt et al., J. Physiol. 490(Pt. 2): 320-336 (1996)) and bitter (Caicedo and Roper, 2002; Caicedeo and Roper, Science 291:1557-60 (2001); Akrabas et al., Science, 242:1047-1050 (1988) (117, 119, 120)) modalities trigger an increase of intracellular calcium concentration in TRCs. Moreover, bitter compounds lead to PTX-sensitive accumulation of inositol triphosphate in TPCs (121, 122). These cells are enriched in classical G protein-signaling effectors such as phospholipase C-β2 (PLCβ2) (18, 23, 26, 124), an enzyme known to be activated by the Gβγ subunit of G proteins belonging to the Gᵢ family (20-24), the type-III inositol trisphosphate receptor (IP3R-III) (123, 124) and a transient receptor potential (trp) channel TRPM5 (53, 72, 78) (FIG. 8). PLCβ2 and TRPM5 are essential for taste perception of sweeteners, bitter substances and amino acids in rodents (18). Collectively, these observations suggest that the major taste transduction pathway in TRCs links α-gustducin to the activation of PLCβ2 and TRPM5, these events ultimately leading to membrane depolarization and taste perception (FIG. 8) (18). We propose that Gβγ subunits released from activated G_{αi} could also contribute to activation of PLCβ2 in TRCs (FIG. 8). Herein, it was shown the mRNAs for PLβ32 and Gαᵢ₋₂ co-exist in the same TRCs and that Gαᵢ₋₂-positive cells also express bitter taste receptors (26). This pathway would directly complement the lack of α-gustducin in mice and would account for the residual responsiveness to bitter compounds and even possibly sweeteners. Confirmation of this signaling pathway can be evaluated in genetically engineered mice lacking α-gustducin in addition to one or more Gαᵢ subunits.

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the claims which follow.

### REFERENCES*

1. Blenis, Proc. Natl Acad. Sci, USA 90: 5889 (1993)
2. Blumer et al., TIBS 19:235 (1994)
3. Camp et al., TIBS 20:117 (1995)
4. Seger et al. FASEB J. 9:726 (1995)
5. David, R. J. TIBS 19:470 (1994)
6. Robinson and Dickenson Eur. J. Pharmacol. 413(2-3):151-61 (2001)
7. Lopaz-Ilasaca, Biochem Pharmacol. 56637:269-77 (1998)
8. Ito et al., FEBS Lett. 368(1):183-7 (1995)
9. Gilbertson et al., Corr. Opin. Neurobiol., 10(4):519-27 (2000)
10. Margolskee, R F, J. Bio., Chem. 277(1):1-4 (2002)
11. Montmayeur et al., Corr. Opin. Neurobiol., 12(4):366-71 (2002)
12. Adler et al., Cell 100(6):693-702 (2000)
13. Matsunami et al., Nature 404(6678):601-4 (2000)
14. Li et al., Proc. Natl Acad Sci., USA 99(7):4692-6 (2002)
15. Nelson et al., Nature (6877):199-202 (2002)
16. Nelson et al., Cell 106(3):381-96 (2001)
17. Wong et al., Nature 381(6585):796-800 (1996)
18. Zhang et al., Cell 112(3):293-301 (2003)
19. Hoffman et al., Nature 397(6716): 259-263 (1999)
20. Li et al., Science 287(54-55):1046-9 (2000)
21. Wu et al., Proc. Natl Acad Sci., USA 90(11):5297-5301 (1993)
22. Katan, Biochem Biophys. Acta 1436(1-2):5-17 (1998)
23. Smrcka et al., J. Biol Chem. 272(24):15045-48 (1993)
24. Rhee et al., J. Biol. Chem. 272(24):15045-8 (1997)
25. Kusakabe et al., Chem. Senses 25(5):525-31 (2000)
26. Asano-Miyoshi Neurosci. Lett. 283(1):64 (2000)
27. He et al., Chem. Senses 27(8):719-27 (2002)
28. Ruiz-Avila et al., Proc. Natl. Acad. Sci., USA 98(15):2868-73 (2001)
29. Harness et al., J. Physiol. 543(Pt. 2):601-614 (2002)
30. Cao et al., Neuroreport 13(10):1321-25 (2002)
31. Roper et al., Ann. Rev. Neurosci. 12:329-53 (1989)
32. Hoon et al., Cell 96:541-551 (1999)
33. Fong, T M Cell Signal. 8(3):217-24 (1996)
34. Baldwin et al., J. Mol. Biol. 272(1):144-64 (1997)
35. Hamm et al., Science 241:832-5 (1988)
36. Osawa et al., J. Biol. Chem. 270:31052-8 (1995)
37. Garcia et al., EMBO 13:4460-9 (1995)
38. Sullivan et al., Nature 330:758-60 (1987)
39. Resenick et al., J. Biol Chem. 269:21519-21525 (1994)
40. West et al., J. Biol Chem., 260:14428-30 (1985)
41. Conklin et al., Nature 363:274-276 (1993)
42. Conklin et al., Mol. Pharmacol. 50:885-890 (1996)
43. Gilchrist et al., J. Biol. Chem. 273:14912-19 (1998)
44. Buck and Axel, Cell 65:175-187 (1991)
45. Stryer, Biochemistry, 3d ed. 1988.
46. Kyte & Doolittle, J. Mol. Biol. 157:103-32 (1982)
47. Oligonucleotides and Analogues a Practical Approach, et. F. Ecksten, Oxford, Univ. Press (1991)
48. Antisense Strategies, Annals of the NY Acad. Sciences, 600 eds. Baserga et al., (NYAS 1992)
49. Milligan, J. Med. Chem. 36:1923-37 (1995)
50. Antisense Research and Applications, (1993, CRC Press)
51. Matag, Toxicol. Appl. Pharmacol. 144:189-197 (1997)
52. Strauss-Soukup, Biochem. 36:8692-8 (1997)
53. Samstag, Antisense Nucl. Acid Drug Dev. 6:153-6 (1996)
54. Batzer et al., Nucl. Acids Rev. 19:5081 (1991)
55. Ohtsuka et al., J. Biol. Chem. 260:2605-8 (1985)
56. Rossolini et al., Mol. Cell. Probes 8:91-98 (1994)
57. Creighton, Proteins, W. H. Freeman and Co. (1984)
58. Schultz and Schimer, Principles of Protein Structure, Springer-Verlog (1979)
59. Harlow & Lane, Antibodies, A. Laboratory Manual (1988)
60. Sambrook et al. (eds.), Molecular Cloning: A Laboratory Manual, Cold Spring Harbor Laboratory Press, Cold Spring Harbor, N.Y.
61. Silhavy, Experiments With Gene Fusions, Cold Spring Laboratory Press, Cold Spring Harbor, N.Y. (1984)
62. Glover & Hanes, DNA Cloning: A Practical Approach, 2^{nd} ed. IRL Press and Oxford University Press, Oxford, N.Y. (1995).
63. Ausubel (ed.), Short Protocols in Mol. Biol., 3^{rd} ed., Wiley, N.Y. (1995)
64. Saqi et al., Bioinformatics 15:521-22 (1999)
65. Barton, Acta Crystallogr. D. Biol. Crystallogr. 54:1139-46 (1998)
66. Henikoff et al., Electrophoresis 21:1700-1706 (2000)
67. Huang et al., Pac. Symp. Biocomp.: 230-241 (2000)
68. Henikoff et al., Adv. Protein Chem. 54:73-97 (2000)
69. Kyte et al., J. Mol. Biol. 157(1):105-32 (1982)
70. Tan et al., J. Immunol. Meth. 232(1-2):87-97 (1998)
71. Vaster et al., Biochem. J. 350:717-22 (2000)
72. “Indirect Mechanisms of Synaptic Transmission,” Chapter 8, From Neuron to Brain (3^{rd} edition) Nichols, J. G. et al., eds., Sinauer Associates, Inc. (1992)
73. McLaughlin et al., Nature 357:563-9 (1992)
74. Ruiz-Avila et al., Nature 376:80-85 (1995)
75. McLaughlin et al., Phys. Behav. 56(6):1157-64 (1994)
76. Yan et al., Am. J. Physiol. Cell Physiol. 280(4):C742-751 (2001)
77. Naim et al., Comp. Biochem Physiol B., 100(3):455-8 (1991)
78. Striem et al., Biochem J. 260(1):121-126 (1989)
79. Kostenis, Trends Pharmacol. Sci., 22(11):560-4 (2001)
80. Chandrashekar et al., Cell 100(6):703-11 (2000)
81. Adamus et al., Vision Res. 31(1):17-31 (1991)
82. Bufe et al., J. Receptor Signal Transduct. Res. 20(2-3):153-166
83. Della Rocca et al., J. Biol. Chem. 272(31):19125-19132 (1997)
84. Della Rocca et al., J. Biol. Chem. 274(2):13978-13984 (1999)
85. Bufe et al., Nat. Genet. 32(3):397-401
86. Yamaguchi et al., Physiol Behav. 49(5):833-841 (1991)
87. Liebmann et al., Cell Signal 13(11):777-785 (2001)
88. Daaka et al., Nature 390:88-91 (1997)
89. Morris and Milbon Physiol Rev. 79(4):1373-1430 (1999)
90. Chin et al., Ann NY Acad Sci. 968:49-64 (2002)
91. Liebmann et al., J. Biol. Chem 271(49):31098-31105 (1996)
92. Robinson-White and Stratekis, Ann. NY Acad. Sci. 968:256-270 (2002)
93. Hanoune and Defer, Ann. Rev. Pharmacol. Toxicol. 41:145-174 (2001)
94. Pierce et al., Oncogene 20(13):1532-1539 (2001)
95. Gutkind, J. S., J. Biol Chem. 273(4):1839-42 (1998)
96. Crespo et al., Nature 369:418-20 (1994)
97. Faure et al., J. Biol Chem. 269(11):7851-9 (1994)
98. Wan et al., J. Biol Chem. 272(27):17209-17215 (1997)
99. Wu et al., Biochem Biophys Acta 1582(1-3):100-6 (2002)
100. Temussi P A, FEBS Lett. 526(1-3):1-4 (2002)
101. Fields, T A and Casey, P J, Biochem J. 321(Pt. 3):561-71 (1997)
102. Offermanns S., Naunyn Schmeideberg, Arch Pharmacol. 360(1)5-13 (1999)
103. Soeder et al., J. Biol. Chem. 274(17):12017-12022 (1999)
104. Alderton et al., J. Biol Chem. 276(16):13452-13460 (2001)
105. Alderton et al., Br. J. Pharmacol. 134(1):6-9 (2001)
106. Kolesnikov and Margolskee, Nature 376:85-8 (1995).
107. Tonosaki et al., Nature 331:354-6 (1988)
108. Avanet et al., Nature 331:351-354 (1988)
109. Cummings and Kinnaman, J. Neurophysiol 70(6):2326-2336 (1993)
110. Cummings and Kinnaman, J. Neurophysiol. 75(3):1236-63 (1996)
111. Uchida and Sato, Chem. Senses 22(3):163-164 (1997)
112. Nakashima and Ninomiya, Cell Physiol Biochem. 9(2):90-98 (1999)
113. Chaudhari and Roper, Ann. NY Acad. Sci. 855:398-406 (1998)
114. Ninomiya et al., J. Nutr. 130(445 Suppl.) 9505-9535 (2000)
115. Hoon et al., Cell 96(4):541-551 (1999)
116. Montmayeur et al., Nat. Neurosci. 4(5):492-498 (2001)
117. Caicedo and Roper, J. Physiol. 544(Pt. 2):501-504 (2002)
118. Bernhardt et al., J. Physiol 490(Pt. 2):325-336 (1996)
119. Caicedo and Roper, Science 291:1557-1560 (2001)
120. Akabas et al., Science 242:1047-1050 (1988)
121. Spielman et al., Physiol Behav. 56660:1149-1153 (1994)
122. Speilman et al., Am J. Physiol. 270(3 Pt. 1) C926-931 (1996)
123. Clapp et al., BMC Neurosci., 21(1):6 (2001)
124. Perez et al., Nat. Neurosci. 5(1-1):1169-1176 (2002)
125. Lush, I. E., and Holland, G. (1988) Genet Res 52(3), 207-212.
126. Kim, U. K, Jorgenson, E., Coon, H., Leppert, M., Risch, N., and Drayna, D. (2003) Science 299(5610), 1221-1225.
127. Ming, D., Ruiz-Avila, L., and Margolskee, R. F. (1998) Proc Natl Acad Sci USA 96(15),8933-8938.
128. Ruiz-Avila, L., Ming, D., and Margolskee, R. F. (2000) Chem Senses 25(4), 361-368.
129. Ogura, T., Margolskee, R. F., and Kinnamon, S. C. (2002) J Neurophysiol 87(6), 3152-3155.
130. Traynor and Nohorski, Mol. Pharmacol. 47:848-854 (1995)
131. Stabbis, et al., Annal. Biochem. 252:115-126 (1997)
132. Dethux et al., J. Exp. Med. 192:15018 (2000)
133. Kenimer & Nirenberg, Mol. Pharmacol. 20:585-591
134. Solomon, et al., Anal. Biochem. 58:541-8 (1974)
135. Horton & Boxendale, Methods Mol. Biol. 41:91-105 (1993)
136. Phospholipid Signalling Protocols, edited by Ian M. Bird. Totowa, N.J., Humana Press, (1998):
137. Rudolph et al., J. Biol. Chem. 274: 11824-11831 (1999)
138. Kikkawa et al., J. Biol. Chem 257:13341 (1982)
139. Pinna & Ruzzenc, Biochem. Biophys Acta 1314:191-225 (1996)
140. Verma et al., Cell 51:513-514 (1987)
141. U.S. Pat. No. 5,519,649
142. Fink et al., 1988, Proc. Natl. Acad. Sci. 85:6662-6666) (1988)
143. Montminy et al., Proc. Natl. Acad. Sci. 83:6682-6686 (1986)
144. Comb et al., Nature 323:353-356 (1986)
145. Short et al., J. Biol. Chem. 261:9721-9726 (1986)
146. Lee et al., Nature 325: 368-372 (1987)
147. Lee et al., Cell 49: 741-752 (1987)
148. Hiscott et al., Mol. Cell. Biol. 13:6231-6240 (1993)
149. Shakhov et al., J. Exp. Med. 171: 35-47 (1990)
150. Liu et al., AIDS Res. Hum. Retroviruses 14: 1509-1519 (1998)
151. Liu et al., AIDS Res. Hum. Retroviruses 14: 1509-1519 (1998)
152. Matsui et al., J. Immunol. 161: 3469-3473 (1998)
153. Schreck & Baeuerle, Mol. Cell. Biol. 10: 1281-1286 (1990)
154. Haskill et al., Cell 65: 1281-1289 (1991)
155. Hafner, 2000, Biosens. Bioelectron. 15: 149-158 (2000)
156. Molecular Cell Biology, Darnell et al, Chapter 16 (1986)
157. Spatola, Chemistry and Biochemistry of Amino Acids, Peptides and Proteins, Vol. 7, pp 267-357, “Peptide Backbone Modifications,” Marcell Dekker, NY (1983)
   * All of the Patents and literature references cited in this application are incorporated by reference in their entireties herein.

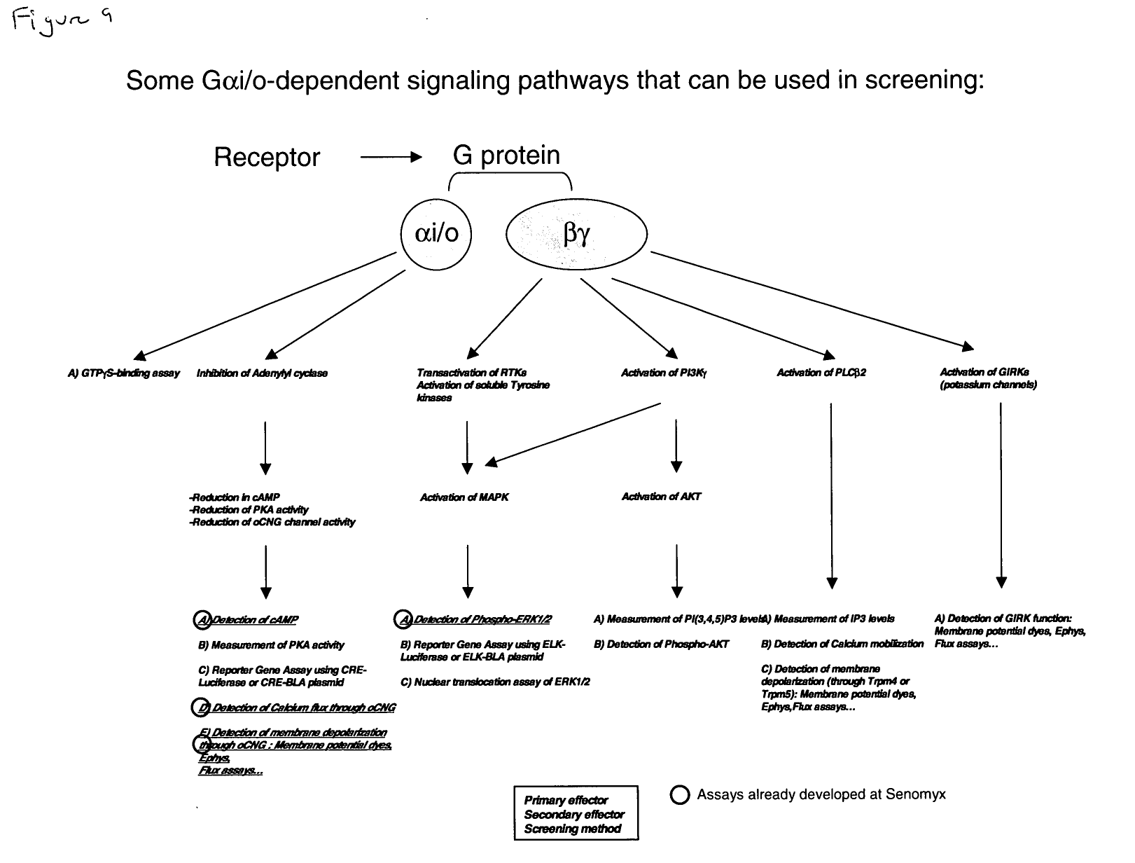

As shown in FIG. 9 and as evidenced by the working examples herein, the present invention embraces any screening assay for detecting T1R or T2R modulators that detects for a compound that affects a Galphai/0-dependent signaling pathway. Exemplary thereof are (i) GTPgammaS-binding assays, (ii) assays that screen for compounds that modulate (inhibit) adenylyl cyclase, (iii) assays that screen for compounds that transactivate RTKs (activate soluble tyrosine kinases); (iv) assays that screen for compounds that activate PI3 Kgamma; (v) assays that screen for compounds that activate PLCbeta2; and (vi) assays that screen for compounds that activate GIRKs (potassium channels).

Assays that detect compounds that inhibit adenylyl cyclase may for example detect a reduction in cAMP, a reduction in PKA activity, a reduction in olfactory cyclic nucleotide gated channel (oCNG) activity (e.g., by detecting calcium flux through oCNG), or by the use of reporter gene assays that use for example CRE-luciferase or CRE-BLA plasmid or assays that detect membrane depolarization through oCNG, e.g. by use of membrane potential dyes, Ephys or flux assays.

Assays that detect RTKs may detect activation of MAPK as described herein such as by the detection of Phospho-ERK1/2; reporter assays that use ELK-Luciferase or ELK-BLA plasmid, or nuclear translocation assays that detect ERK1/2.

Assays that detect PI3 Kgamma may detect activation of ALK by for example measuring PI (3,4,5) P3 levels, or by detecting Phospho-AKT.

Assays that detect PLCbeta2 may detect IP3 levels, detect calcium mobilization, or detect membrane depolarization (through Trpm4 or Trpm5 channels) using membrane potential dyes, Ephys or flux assays.

Assays that detect GIRKs (potassium channels) may detect GIRK function e.g., using membrane potential dyes, Ephys or flux assays.

Means and kits for effecting such assays are known in the art. For example see Lundholt et al., J Biomolecular Screening, 2005:20-29; Fan et al., Cell Notes Issue 13:5-7(2005); the beta lactamase reporter system provided by Invitrogen, the pTET-ELK vector system available from Clontech Laboratories Inc, the IP3 fluorescence polarization assay provided by DiscoveRx; and the Phospho-Akt assay provided by HRTF In Vitro Technologies-HTRF and the PI 3-kinases assay provided by Echelon Biosciences Inc, all incorporated by reference in their entirety herein.

1. A method for identifying a potential T1R or T2R modulatory compound comprising:
(i) contacting a eukaryotic cell that expresses at least one T1R or T2R taste receptor and a Galphai protein that functionally couples thereto with a putative modulator of said at least one T1R or T2R taste receptor: and
(ii) detecting whether said compound modulates the activity of said at least one T1R or T2R based on whether it has an assayable affect on a Gi signalling pathway in said cell.

2. The method of claim 1 wherein said at least one T1R or T2R is a human T1R or T2R.

3. The method of claim 1 wherein said cell expresses at least one human T2R.

4. The method of claim 1 wherein said cell expresses at least one human T1R.

5. The method of claim 4 wherein said cell expresses human T1R1 and human T1R3.

6. The method of claim 4 wherein said cell expresses human T1R2 and human T1R3.

7. The method of claim 1 wherein the eukaryotic cell is selected from the group consisting of an amphibian cell, yeast, mammalian cell, and insect cell.

8. The method of claim 1 wherein the cell is selected from a Hela cell, CHO cell, BHK cell, HEK293 cell and a Xenopus oocyte.

9. The method of claim 8 wherein the cell is a HEK293 cell.

10. The method of claim 1 wherein the Gi signaling pathway results in the activation of P13 Kgamma.

11. The method of claim 10 wherein said activation is detected by measuring PI(3,4,5)P3 levels.

12. The method of claim 10 wherein said activation is detected by assaying for changes in phospo-AKT levels.

13. The method of claim 1 which detects Gi mediated activation of PLCbeta2.

14. The method of claim 13 wherein said activation is detected by assaying for changes in IP3 levels.

15. The method of claim 13 which detects for changes in calcium mobilization in said cell.

16. The method of claim 13 which detects for membrane depolarization.

17. The method of claim 16 wherein membrane depolarization is mediated by Trpm4 or Trpm5.

18. The method of claim 16 which uses a membrane potential dye, Ephys or a flux assay.

19. The method of claim 1 which detects for the effect of said compound on the activation of a GIRK (potassium channel).

20. The method of claim 19 wherein said effect on GIRK function is detected using a membrane potential dye, Ephys or a flux assay.
